(19) United States
(12) Patent Application Publication
(10) Pub. No.: US 2021/0188897 A1
(43) Pub. Date: Jun. 24, 2021

(54) NEUROACTIVE STEROIDS AND COMPOSITIONS AND METHODS THEREOF

(71) Applicant: Sparx Therapeutics, Inc., Mount Prospect, IL (US)

(72) Inventors: Qian Chen, Chicago, IL (US); Guidong Zhu, Gurnee, IL (US); Hongyu Zhao, Libertyville, IL (US)

(21) Appl. No.: 17/099,568

(22) Filed: Nov. 16, 2020

Related U.S. Application Data
(60) Provisional application No. 62/944,006, filed on Dec. 5, 2019.

(51) Int. Cl. C07J 41/00; C07J 11/00; C07J 9/00; C07J 51/00; C07J 53/00; A61K 45/06

(52) U.S. Cl. CPC C07J 41/0094; C07J 11/00; A61K 45/06; C07J 51/00; C07J 53/002; C07J 9/005

(57) ABSTRACT

The invention provides novel neuroactive steroids and pharmaceutical compositions thereof, as well as methods of their preparation and use, in therapy of various diseases and conditions, for example, various neurological or brain diseases.

### PRIORITY CLAIMS AND RELATED APPLICATIONS

This application claims the benefit of priority to U.S. Provisional Application No. 62/944,006, filed Dec. 5, 2019, the entire content of which is incorporated herein by reference for all purposes.

### TECHNICAL FIELDS OF THE INVENTION

The invention generally relates to pharmaceuticals and therapeutic methods. More particularly, the invention provides novel neuroactive steroids and pharmaceutical compositions thereof, as well as methods of their preparation and use in treating various diseases and conditions.

### BACKGROUND OF THE INVENTION

Healthy brain function depends on a precise regulation of neuronal activity. Many psychiatric and neurological diseases are associated with spatial and temporal imbalance of excitatory and inhibitory neurotransmission in brain. Gamma-Amino butyric acid (GABA) is a predominant inhibitory neurotransmitter in the mammalian central nervous system and is essential for the overall balance between neuronal excitation and inhibition (Markram, H. et al., Nat Rev Neurosci. 2004, 5, 793-807). GABAergic neurons are present throughout all levels in the nervous system and represent between 20 and 40% of all neurons depending on brain region. GABA exerts its effects by activation of two different classes of receptors, the ionotropic GABA_{A} receptors (GABA_{A}Rs) and the metabotropic GABA_{B} receptors (Sivilotti, L. et al., Prog. Neurobiol. 1991, 36, 35-92).

GABA_{A} receptors mediate fast inhibitory neurotransmission in the central nervous system (CNS). GABA_{A}Rs are pentameric membrane-bound proteins that are members of the Cys-loop superfamily of ligand-gated ion channels. There are 6 types of α unit, 3 types of β unit, 3 types of γ unit, along with δ, ε, π, θ and three types of ρ subunits (Rudolph, U. et al., Trends Pharmacol. Sci. 2001, 22, 188-194). These units can potentially form many heteropentameric GABA_{A}Rs and the exact number of GABA_{A}Rs is currently unknown. GABA_{A}Rs comprising γ2, α1-3 and βx subunits with α-β-γ-α-β arrangement (counterclockwise looking from the intracellular side) are the most common type of receptors at synaptic sites and as such are primarily responsible for mediating phasic inhibition (Farrant, M. et al., Nat. Rev. Neurosci. 2005, 6, 215-229). GABA_{A}Rs comprising primarily α4/6, βx subunits combined with a δ subunit are enriched at extrasynaptic sites, responsible for mediating tonic inhibition (Mody, L., Neurochem. Res. 2001, 26, 907-913). The different subunit combination and localization determines the biophysical and pharmacological functions of the assembled GABA_{A}Rs.

Human GWAS studies have demonstrated that genetic mutations in GABA_{A}Rs play a role in multiple neurological and neuropsychiatric disorders, including schizophrenia, bipolar and epilepsies (Cherlyn, S. Y. et al., Neurosci. Biobehav. Rev. 2010, 34, 958-977; Macdonald, R. L. et al. J. Physiol. 2010, 34, 958-977; Macdonald, R. L. et al., J. Physiol. 2010, 588, 1861-1869). In many cases these conditions are precipitated or exacerbated by chronic stress, which also causes extensive alteration of GABA transmission and its receptor expression, especially GABA_{A}Rs (Vaiva, G. et al., Biol. Psychiatry 2004, 55, 250-254; Merali, Z. et al., J. Neurosci. 2004, 24, 1478-1485; Poulter, M. O. et al., Biol. Psychiatry 2008, 64, 645-652; Geuze, E. et al., Mol. Psychiatry 2008, 13, 74-83).

Most neurodevelopmental disorders (autism, Angelman, Rett and Fragile X syndromes etc.) have overlapping symptoms such as anxiety, sleep dysfunction, cognitive deficits and epilepsy, and have been linked to a decreased inhibitory tone leading to an excitatory/inhibitory (E/I) imbalance (Rubenstein, J. L. et al., Genes Brain Behav. 2003, 2, 255-267; Braudeau, J. et al., J. Psychopharmacol. 2011, 25, 1030-1042). GABAergic deficits have been proposed to play a role in the pathophysiology of epilepsy, depression, schizophrenia, bipolar disease, and neurodegenerative diseases such as Alzheimer's and Parkinson's diseases. The alterations in both synaptic and extrasynaptic GABA_{A}Rs have also been implicated in schizophrenia (Guidotti, A. et al., Psychopharmacology 2005, 180, 191-205; Damgaard, T. et al., Psychopharmacology 2011, 214, 403-413).

The role of GABA_{A}Rs in depression has generated significant interest because of the several compounds exhibiting antidepressant/mood-stabilizing properties that also have effects on GABAergic transmission (Kalueff, A. V. et al., Depress Anxiety 2007, 24, 495-517). Deficits in GABAergic inhibition have been proposed to play a critical role in the pathophysiology of depression (Luscher, B. et al., Mol. Psychiatry 2011, 16, 383-406; Maguire, J. et al., Neuron 2008, 59, 207-213). However, increasing GABA levels in brain is accompanied by numerous detrimental side effects. Selective targeting on synaptic and extrasynaptic GABA_{A} receptor subtypes may eliminate the need to modulate GABA levels and reduce side effects.

Neurosteroids are biosynthesized in the brain from cholesterol or through the conversion of peripheral steroids which have potent and selective effects on the GABA_{A} receptors (Beshir, K. et al., Front. Neurol. 2017, 8, Article 442). The ovarian hormone progesterone and its metabolites such as allopregnanolone have shown profound effects on brain excitability (Lambert, J. et al., Trends Pharmacol. Sci. 1987, 8, 224-227). Epilepsy can be considered a result of pathologically high neuronal excitability and low levels of progesterone and its metabolites was associated with higher seizure frequency in female epileptics (Laidlaw, J., Lancet, 1956, 1235-1237; Rosciszewska et al. J. Neurol. Neurosurg. Psych. 1986, 49, 47-51). The levels of these hormones fluctuate during the menstrual cycle and coincide with certain physical effects associated with premenstrual syndrome, including stress, anxiety, and migraine headaches (Dalton, K., Premenstrual Syndrome and Progesterone Therapy, 2nd edition, Chicago Yearbook, Chicago (1984)). In primary generalized petit mal epilepsy patients, the temporal incidence of seizures correlated with the incidence of the symptoms of premenstrual syndrome (Backstrom, T. et al., J. Psychosom. Obstet. Gynaecol. 1983, 2, 8-20) and deoxycorticosterone has been used to treat subjects whose epileptic spells correlate with their menstrual cycles (Aird, R. B. et al. J. Am. Med. Soc. 1951, 145, 715-719).

Low levels of progesterone and its metabolites such as allopregnanolone are also associated with postpartum depression (PPD). The symptoms of PPD include depression, rumination, anxiety and irritability, and hospitalization is required in severe cases. Progesterone levels rise during pregnancy and decrease dramatically after birth, coinciding with the onset of PPD. Allopregnanolone modulates neuronal excitability through direct action on synaptic and extrasynaptic GABA_{A}Rs (Belelli, D. et al. J. Neuroscience, 2009, 29, 12757-12763). Inappropriate response of GABA_{A}Rs to the changes in allopregnanolone levels after birth may play a role in triggering PPD (Maguire, J. et al, Neuron, 2008, 59, 207-213). Allopregnanolone has demonstrated efficacy in preclinical anxiety models (Schule, C. et al. Progress in Neurobiology, 2014, 113, 79-87) and elevated allopregnanolone levels may protect against depressed mood during pregnancy (Hellgren, C. et al, Neuropsychobiology, 2014, 69, 147-153). An infusion formulation of allopregnanolone (Zulresso) has demonstrated clinical efficacy compared to placebo in PPD patients (Kanes, S. et al. The Lancet. 2017, 390, 480-489) and was approved for the treatment of PPD by the FDA in 2019. However, Zulresso is only administered through intravenous infusion. An orally available compound Zuranolone also showed efficacy in clinical trials, but this compound caused upper respiratory infections. There is a need for orally available GABA_{A} receptor potentiators with improved tolerability.

Allopregnanolone levels decline with age and the onset of neurodegenerative diseases such as Alzheimer's disease (Marx, C. E. et al. Biol. Psych. 2006, 60, 1287-1294.). Allopregnanolone was found to promote neurogenesis in the hippocampal subgranular zone and reverse learning and memory deficits in preclinical models (Wang, J. M. et al. Proc. Natl. Acad. Sci. 2010, 107, 6498-6503). Allopregnanolone increased survival of newly generated neurons and simultaneously reduced AO pathology in rodents (Chen, S. et al. PloS one, 2011, 68, e24293). Taken together, these results suggest that allopregnanolone and its derivatives may alleviate the symptoms and/or halt the progression of Alzheimer's disease.

Positive allosteric modulators (PAM) of GABA_{A}Rs such as benzodiazepines and Z-drugs have been the mainstay of insomnia treatment. However, these medicines have serious side effects such as next-day sleepiness, cognitive impairment, dependency, rebound, and withdrawal liabilities (Atkin, T. et al. Pharmacolog. Rev. 2018, 70, 197-245.). Next-day sleepiness is the most common side effect, which is especially troublesome for elderly patients since falling significantly increases death risk in seniors. Next-day sleepiness is linked to the long half-life of several of these drugs. The American Association of Sleeping Medicine recommends only three drugs for sleep onset insomnia, all of which have short half-life in humans (t_{1/2}<3 hours). Pharmacokinetic properties are key optimization parameters in insomnia drug discovery.

There is an urgent need for novel therapeutics and treatment methods for various neurological or brain diseases that provide improved clinical effectiveness with reduced side effects.

### SUMMARY OF THE INVENTION

The invention provides novel compounds, and compositions and methods thereof, that selectively modulate the activity of GABA_{A} receptors, for example, that contain δ or γ subunits, which encompasses receptors composed of δ or γ subunits in combination with α or β subunits (i.e., α/β/δ or α/β/γ receptors). These novel therapeutics and treatment methods are useful in treating various neurological or brain diseases and offer improved clinical outcome and reduced side effects.

In one aspect, the invention generally relates to a compound having the structural formula (I):

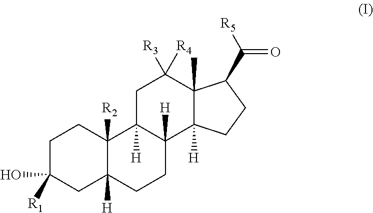

wherein

R₁ is H or a substituted or unsubstituted C₁-C₆ alkyl;

R₂ is H or a substituted or unsubstituted C₁-C₆ alkyl;

each of R₃ and R₄ is independently selected from the group consisting of H, halogen, a substituted or unsubstituted C₁-C₆ alkyl, optionally R₃ and R₄, along with the carbon to which they are attached, may form an exocyclic double bond, or a C₃-C₁₈-membered ring optionally substituted with one or more substituents selected from the group consisting of halogen, OH, CN, C₁-C₅ alkyl, and O—C₁-C₅ alkyl;

R₅ is OR′ or a C₁-C₆ alkyl optionally substituted with heterocyclic or heterobicyclic group, which is optionally substituted with one or more of CN, OH, halogen, a substituted or unsubstituted C₁-C₆ alkyl; provided that, if each of R₃ and R₄ is H, R₅ is a C₁ alkyl substituted with a heterocyclic or heterobicyclic group, which is optionally substituted with C═O(NR^{f}R^{g}), C(R^{f})(R^{g})(OR^{h}), or OR^{h}, wherein each of R′, R^{f} and R^{g} is independently selected from the group consisting of H, a substituted or unsubstituted C₁-C₆ alkyl, and R^{h} is (CH₂CH₂O)ₙCH₃ or CH₂O(CH₂CH₂O)ₙCH₃, wherein n is 1, 2, 3, 4 or 5,

or a pharmaceutically acceptable form or an isotope derivative thereof.

In another aspect, the invention generally relates to a pharmaceutical composition comprising a compound disclosed herein, effective to treat or reduce one or more diseases or disorders, in a mammal, including a human, and a pharmaceutically acceptable excipient, carrier, or diluent.

In another aspect, the invention generally relates to a pharmaceutical composition comprising a compound having the structural formula (I)

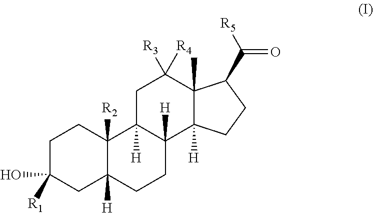

wherein

R₁ is H or a substituted or unsubstituted C₁-C₆ alkyl;

R₂ is H or a substituted or unsubstituted C₁-C₆ alkyl;

each of R₃ and R₄ is independently selected from the group consisting of H, halogen, a substituted or unsubstituted C₁-C₆ alkyl, optionally R₃ and R₄, along with the carbon to which they are attached, may form an exocyclic double bond, or a C₃-C₁₈-membered ring optionally substituted with one or more substituents selected from the group consisting of halogen, OH, CN, C₁-C₅ alkyl, and O—C₁-C₅ alkyl;

R₅ is OR′ or a C₁-C₆ alkyl optionally substituted with heterocyclic or heterobicyclic group, which is optionally substituted with one or more of CN, OH, halogen, a substituted or unsubstituted C₁-C₆ alkyl; provided that, if each of R₃ and R₄ is H, R₅ is a C₁ alkyl substituted with a heterocyclic or heterobicyclic group, which is optionally substituted with C═O(NR^{f}R^{g}), C(R^{f})(R^{g})(OR^{h}), or OR^{h}, wherein each of R′, R^{f} and R^{g} is independently selected from the group consisting of H, a substituted or unsubstituted C₁-C₆ alkyl, and R^{h} is (CH₂CH₂O)ₙCH₃ or CH₂O(CH₂CH₂O)ₙCH₃, wherein n is 1, 2, 3, 4 or 5,

or a pharmaceutically acceptable form or an isotope derivative thereof, effective to treat or reduce one or more diseases or disorders, in a mammal, including a human, and a pharmaceutically acceptable excipient, carrier, or diluent.

In yet another aspect, the invention generally relates to a unit dosage form comprising a pharmaceutical composition disclosed herein.

In yet another aspect, the invention generally relates to a method for treating or reducing a disease or disorder, comprising administering to a subject in need thereof a pharmaceutical composition comprising a compound having the structural formula (I):

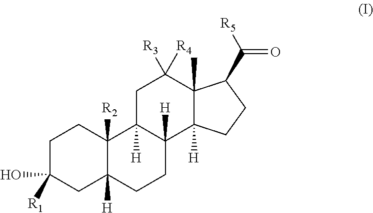

wherein

R₁ is H or a substituted or unsubstituted C₁-C₆ alkyl;

R₂ is H or a substituted or unsubstituted C₁-C₆ alkyl;

each of R₃ and R₄ is independently selected from the group consisting of H, halogen, a substituted or unsubstituted C₁-C₆ alkyl, optionally R₃ and R₄, along with the carbon to which they are attached, may form an exocyclic double bond, or a C₃-C₁₈-membered ring optionally substituted with one or more substituents selected from the group consisting of halogen, OH, CN, C₁-C₅ alkyl, and O—C₁-C₅ alkyl;

R₅ is OR′ or a C₁-C₆ alkyl optionally substituted with heterocyclic or heterobicyclic group, which is optionally substituted with one or more of CN, OH, halogen, a substituted or unsubstituted C₁-C₆ alkyl; provided that, if each of R₃ and R₄ is H, R₅ is a C₁ alkyl substituted with a heterocyclic or heterobicyclic group, which is optionally substituted with C═O(NR^{f}R^{g}), C(R^{f})(R^{g})(OR^{h}), or OR^{h}, wherein each of R′, R^{f} and R^{g} is independently selected from the group consisting of H, a substituted or unsubstituted C₁-C₆ alkyl, and R^{h} is (CH₂CH₂O)ₙCH₃ or CH₂O(CH₂CH₂O)ₙCH₃, wherein n is 1, 2, 3, 4 or 5,

or a pharmaceutically acceptable form or an isotope derivative thereof, effective to treat or reduce one or more of PPD, major depressive disorder (MDD or depression), insomnia, sleep apnea, restless legs syndrome, and narcolepsy, emotional disorders, depression, schizophrenia, bipolar disorder, obsessive-compulsive disorder, and other anxiety disorders, behavioral and pharmacological syndrome of dementia, and neurodegenerative diseases, or a related disease or disorder, in a mammal, including a human.

In yet another aspect, the invention generally relates to a method for treating or reducing a disease or disorder, comprising administering to a subject in need thereof a pharmaceutical composition comprising a compound disclosed herein, wherein the disease or disorder is one or more of PPD, depression, insomnia, sleep apnea, restless legs syndrome, and narcolepsy, emotional disorders, depression, schizophrenia, bipolar disorder, obsessive-compulsive disorder, and other anxiety disorders, behavioral and pharmacological syndrome of dementia, neurodegenerative diseases, or a related disease or disorder.

In yet another aspect, the invention generally relates to use of a compound disclosed herein, and a pharmaceutically acceptable excipient, carrier, or diluent, in preparation of a medicament for treating a disease or disorder.

### BRIEF DESCRIPTION OF THE DRAWINGS

FIG. 1a and FIG. 1b show certain exemplary data regarding brain (FIG. 1a) and plasma (FIG. 1b) exposure experiments in mice.

FIG. 2 shows certain exemplary data regarding locomotor functions.

FIG. 3 shows certain exemplary data regarding a forced swim test in 5XFAD and wild type mice.

FIG. 4 shows certain exemplary data regarding a tail suspension test in 5XFAD and wild type mice.

FIG. 5 shows certain exemplary data regarding a novel object recognition (NOR) test in 5XFAD and wild type mice.

FIG. 6 shows certain exemplary data regarding a social recognition test in 5XFAD and wild type mice.

### DEFINITIONS

Unless defined otherwise, all technical and scientific terms used herein have the same meaning as commonly understood by one of ordinary skill in the art to which this invention belongs. General principles of organic chemistry, as well as specific functional moieties and reactivity, are described in “Organic Chemistry”, Thomas Sorrell, University Science Books, Sausalito: 2006.

As used herein, “at least” a specific value is understood to be that value and all values greater than that value.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

As used herein, the term “administration” of a disclosed compound encompasses the delivery to a subject of a compound as described herein, or a prodrug or other pharmaceutically acceptable form thereof, using any suitable formulation or route of administration, as discussed herein.

The terms “disease”, “disorder” and “condition” are used interchangeably unless indicated otherwise.

As used herein, the terms “effective amount” or “therapeutically effective amount” refer to that amount of a compound or pharmaceutical composition described herein that is sufficient to effect the intended application including, but not limited to, disease treatment, as illustrated below.

In some embodiments, the amount is that effective for stop the progression or effect reduction of an inflammatory disease or disorder. In some embodiments, the amount is that effective for stop the progression or effect reduction of an immune system disorders. In some embodiments, the amount is that effective to stop the progression or effect reduction of an autoimmune disease or disorder. In some embodiments, the amount is that effective for stop the progression or effect reduction of a cardiovascular disease or disorder. In some embodiments, the amount is that effective for detectable killing or inhibition of the growth or spread of cancer cells; the size or number of tumors; or other measure of the level, stage, progression or severity of the cancer. In some embodiments, the amount is that effective for stop the progression or effect reduction of PPD, depression, insomnia, sleep apnea, restless legs syndrome, and narcolepsy, emotional disorders, depression, schizophrenia, bipolar disorder, obsessive-compulsive disorder, and other anxiety disorders, behavioral and pharmacological syndrome of dementia, or neurodegenerative diseases. In some embodiments, the amount is that effective for stop the progression or effect reduction of Parkinson's disease (PD). In some embodiments, the amount is that effective for stop the progression or effect reduction of Alzheimer's disease (AD).

The therapeutically effective amount can vary depending upon the intended application, or the subject and disease condition being treated, e.g., the desired biological endpoint, the pharmacokinetics of the compound, the disease being treated, the mode of administration, and the weight and age of the patient, which can readily be determined by one of ordinary skill in the art. The term also applies to a dose that will induce a particular response in target cells, e.g., reduction of cell migration. The specific dose will vary depending on, for example, the particular compounds chosen, the species of subject and their age/existing health conditions or risk for health conditions, the dosing regimen to be followed, the severity of the disease, whether it is administered in combination with other agents, timing of administration, the tissue to which it is administered, and the physical delivery system in which it is carried.

The term “optionally substituted” is understood to mean that a given chemical moiety (e.g. an alkyl group) can (but is not required to) be bonded other substituents (e.g. heteroatoms). For instance, an alkyl group that is optionally substituted can be a fully saturated alkyl chain (i.e. a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups, but does not necessarily have any further functional groups. Suitable substituents used in the optional substitution of the described groups include, without limitation, halogen, oxo, CN, —COOH, —CH₂CN, —O—C₁-C₆ alkyl, C₁-C₆ alkyl, —OC₁-C₆ alkenyl, —OC₁-C₆ alkynyl, —C₁-C₆ alkenyl, —C₁-C₆ alkynyl, —OH, —OP(O)(OH)₂, —OC(O)C₁-C₆ alkyl, —C(O)C₁-C₆ alkyl, —OC(O)OC₁-C₆ alkyl, NH₂, NH(C₁-C₆ alkyl), N(C₁-C₆ alkyl)₂, —NHC(O)C₁-C₆ alkyl, —C(O)NHC₁-C₆ alkyl, —S(O)₂—C₁-C₆ alkyl, —S(O)NHC₁-C₆ alkyl, and S(O)N(C₁-C₆ alkyl)₂.

The term “tautomers” refers to a set of compounds that have the same number and type of atoms, but differ in bond connectivity and are in equilibrium with one another. A “tautomer” is a single member of this set of compounds. Typically, a single tautomer is drawn but it is understood that this single structure is meant to represent all possible tautomers that might exist. Examples include enol-ketone tautomerism. When a ketone is drawn it is understood that both the enol and ketone forms are part of the invention.

As used herein, a “pharmaceutically acceptable form” of a disclosed compound includes, but is not limited to, pharmaceutically acceptable salts, esters, hydrates, solvates, isomers, prodrugs, and isotopically labeled derivatives of disclosed compounds. In one embodiment, a “pharmaceutically acceptable form” includes, but is not limited to, pharmaceutically acceptable salts, esters, isomers, prodrugs and isotopically labeled derivatives of disclosed compounds. In some embodiments, a “pharmaceutically acceptable form” includes, but is not limited to, pharmaceutically acceptable salts, esters, stereoisomers, prodrugs and isotopically labeled derivatives of disclosed compounds.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively. Pharmaceutically acceptable salts derived from appropriate bases include alkali metal, alkaline earth metal, ammonium and N⁺(C₁₋₄ alkyl)⁴ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt can be chosen from ammonium, potassium, sodium, calcium, and magnesium salts.

In certain embodiments, the pharmaceutically acceptable form is a pharmaceutically acceptable ester. As used herein, the term “pharmaceutically acceptable ester” refers to esters that hydrolyze in vivo and include those that break down readily in the human body to leave the parent compound or a salt thereof. Such esters can act as a prodrug as defined herein. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfinic acids, sulfonic acids and boronic acids. Examples of esters include formates, acetates, propionates, butyrates, acrylates and ethylsuccinates. The esters can be formed with a hydroxy or carboxylic acid group of the parent compound.

In certain embodiments, the pharmaceutically acceptable form is a “solvate” (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In certain embodiments, the pharmaceutically acceptable form is a prodrug. As used herein, the term “prodrug” (or “pro-drug”) refers to compounds that are transformed in vivo to yield a disclosed compound or a pharmaceutically acceptable form of the compound. A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs can increase the bioavailability of the compound when administered to a subject (e.g., by permitting enhanced absorption into the blood following oral administration) or which enhance delivery to a biological compartment of interest (e.g., the brain or lymphatic system) relative to the parent compound. Exemplary prodrugs include derivatives of a disclosed compound with enhanced aqueous solubility or active transport through the gut membrane, relative to the parent compound.

The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. Treatment is aimed to obtain beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compounds and/or compositions can be administered to a patient at risk of developing a particular disease, or to a patient reporting one or more of the physiological symptoms of a disease, even though a diagnosis of this disease may not have been made. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

As used herein, the term “therapeutic effect” refers to a therapeutic benefit and/or a prophylactic benefit as described herein. A prophylactic effect includes delaying or eliminating the appearance of a disease or condition, delaying or eliminating the onset of symptoms of a disease or condition, slowing, halting, or reversing the progression of a disease or condition, or any combination thereof.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

Solvates and polymorphs of the compounds of the invention are also contemplated herein. Solvates of the compounds of the present invention include, for example, hydrates.

As used herein, the term an “isolated” or “substantially isolated” molecule (such as a polypeptide or polynucleotide) is one that has been manipulated to exist in a higher concentration than in nature or has been removed from its native environment. For example, a subject antibody is isolated, purified, substantially isolated, or substantially purified when at least 10%, or 20%, or 40%, or 50%, or 70%, or 90% of non-subject-antibody materials with which it is associated in nature have been removed. For example, a polynucleotide or a polypeptide naturally present in a living animal is not “isolated,” but the same polynucleotide or polypeptide separated from the coexisting materials of its natural state is “isolated.” Further, recombinant DNA molecules contained in a vector are considered isolated for the purposes of the present invention. Isolated RNA molecules include in vivo or in vitro RNA replication products of DNA and RNA molecules. Isolated nucleic acid molecules further include synthetically produced molecules. Additionally, vector molecules contained in recombinant host cells are also isolated. Thus, not all “isolated” molecules need be “purified.”

As used herein, the term “purified” when used in reference to a molecule, it means that the concentration of the molecule being purified has been increased relative to molecules associated with it in its natural environment, or environment in which it was produced, found or synthesized. Naturally associated molecules include proteins, nucleic acids, lipids and sugars but generally do not include water, buffers, and reagents added to maintain the integrity or facilitate the purification of the molecule being purified. According to this definition, a substance may be 5% or more, 10% or more, 20% or more, 30% or more, 40% or more, 50% or more, 60% or more, 70% or more, 80% or more, 90% or more, 95% or more, 98% or more, 99% or more, or 100% pure when considered relative to its contaminants.

Definitions of specific functional groups and chemical terms are described in more detail below. When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “acyl” as used herein, means an alkyl group, as defined herein, appended to the parent molecular moiety through a carbonyl group, as defined herein. Representative examples of acyl include, but are not limited to, acetyl, 1-oxopropyl, 2,2-dimethyl-1-oxopropyl, 1-oxobutyl, and 1-oxopentyl.

The term “acyloxy” as used herein, means an acyl group, as defined herein, appended to the parent molecular moiety through an oxygen atom. Representative examples of acyloxy include, but are not limited to, acetyloxy, propionyloxy, and isobutyryloxy.

The term “Alkenyl” refers to a straight or branched chain unsaturated hydrocarbon containing 2-12 carbon atoms. The “alkenyl” group contains at least one double bond in the chain. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkenylene” means a divalent group derived from a straight or branched chain hydrocarbon of from 2 to 10 carbon atoms containing at least one double bond. The alkenylene is optionally substituted with 1 or 2 substituents selected from the group consisting of aryl and hydroxy. Representative examples of alkenylene include, but are not limited to, —CH═CH—, —CH═CH₂CH₂—, —CH═CH₂CH(Ph)-, —CH═C(CH₃)CH₂—, and —CH═C(CH₃)CH₂CH(OH)CH₂—.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkoxy” as used herein, means an alkoxy group, as defined herein, appended to the parent molecular moiety through another alkoxy group, as defined herein. Representative examples of alkoxyalkoxy include, but are not limited to, tert-butoxymethoxy, 2-ethoxyethoxy, 2-methoxyethoxy, and methoxymethoxy.

The term “alkoxyalkyl” as used herein, means an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl” as used herein, means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxycarbonylalkyl” as used herein, means an alkoxycarbonyl group, as defined herein, appended to the parent molecular moiety through an alkyl group, as defined herein. Representative examples of alkoxycarbonylalkyl include, but are not limited to, 3-methoxycarbonylpropyl, 4-ethoxycarbonylbutyl, and 2-tert-butoxycarbonylethyl.

As used herein, the term “alkyl” refers to a straight or branched hydrocarbon chain radical consisting solely of carbon and hydrogen atoms, containing no unsaturation, having from one to ten carbon atoms (e.g., C₁₋₁₀ alkyl). Whenever it appears herein, a numerical range such as “1 to 10” refers to each integer in the given range; e.g., “1 to 10 carbon atoms” means that the alkyl group can consist of 1 carbon atom, 2 carbon atoms, 3 carbon atoms, etc., up to and including 10 carbon atoms, although the present definition also covers the occurrence of the term “alkyl” where no numerical range is designated. In some embodiments, “alkyl” can be a C₁₋₆ alkyl group. In some embodiments, alkyl groups have 1 to 10, 1 to 8, 1 to 6, or 1 to 3 carbon atoms. Representative saturated straight chain alkyls include, but are not limited to, -methyl, -ethyl, -n-propyl, -n-butyl, -n-pentyl, and -n-hexyl; while saturated branched alkyls include, but are not limited to, -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, and the like. The alkyl is attached to the parent molecule by a single bond. Unless stated otherwise in the specification, an alkyl group is optionally substituted by one or more of substituents which independently include: acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃, —OR^{a}, —SR^{a}, —OC(O)—R^{a}, —N(R^{a})₂, —C(O)Rₐ, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜN(R^{a})₂ (where t is 1 or 2), —P(═O)(R^{a})(R^{a}), or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties can be optionally substituted as defined herein. In a non-limiting embodiment, a substituted alkyl can be selected from fluoromethyl, difluoromethyl, trifluoromethyl, 2-fluoroethyl, 3-fluoropropyl, hydroxymethyl, 2-hydroxyethyl, 3-hydroxypropyl, benzyl, and phenethyl.

The term “-alkylaryl” refers to aryl groups connected to an adjacent C₁-C₆ alkyl wherein the linkage is located at the alkyl end. Accordingly, groups such as benzyl, phenylethyl, or mesitylenyl constitute exemplary representatives of alkylaryl of the present invention.

The term “alkylcarbonylalkyl” as used herein, means an alkylcarbonyl group, as defined herein, appended to the parent molecular moiety through an alkyl group, as defined herein. Representative examples of alkylcarbonylalkyl include, but are not limited to, 2-oxopropyl, 3,3-dimethyl-2-oxopropyl, 3-oxobutyl, and 3-oxopentyl.

The term “alkylcarbonyloxy” as used herein, means an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy.

The term “alkylenyl” as herein defined refers to groups of general formula —(CH₂)ₙ— where n is an integer from 1 to 6. Suitable examples of alkylenyl groups include methylenyl, ethylenyl, and propylenyl.

The term “alkylthio” as used herein, means an alkyl group, as defined herein, appended to the parent molecular moiety through a sulfur atom. Representative examples of alkylthio include, but are not limited, methylthio, ethylthio, tert-butylthio, and hexylthio.

The term “alkylthioalkyl” as used herein, means an alkylthio group, as defined herein, appended to the parent molecular moiety through an alkyl group, as defined herein.

The term “amido” as used herein, means an amino, alkylamino, or dialkylamino group appended to the parent molecular moiety through a carbonyl group, as defined herein. Representative examples of amido include, but are not limited to, aminocarbonyl, methylaminocarbonyl, dimethylaminocarbonyl, and ethylmethylaminocarbonyl.

The term “alkynyl” refers to a straight or branched chain unsaturated hydrocarbon containing 2-12 carbon atoms. The “alkynyl” group contains at least one triple bond in the chain. Examples of alkynyl groups include ethynyl, propargyl, n-butynyl, iso-butynyl, pentynyl, or hexynyl.

Unless otherwise specifically defined, the term “aryl” refers to cyclic, aromatic hydrocarbon groups that have 1 to 2 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, H, halogen, —O—C₁-C₆ alkyl, C₁-C₆ alkyl, —C₁-C₆ alkenyl, —OC₁-C₆ alkynyl, —C₁-C₆ alkenyl, —C₁-C₆ alkynyl, —OH, —OP(O)(OH)₂, —OC(O)C₁-C₆ alkyl, —C(O)C₁-C₆ alkyl, —OC(O)OC₁-C₆alkyl, NH₂, NH(C₁-C₆ alkyl), N(C₁-C₆ alkyl)₂, —S(O)₂—C₁-C₆ alkyl, —S(O)NHC₁-C₆alkyl, and S(O)N(C₁-C₆ alkyl)₂. The substituents can themselves be optionally substituted. Furthermore, when containing two fused rings the aryl groups herein defined may have an unsaturated or partially saturated ring fused with a fully unsaturated ring. Exemplary ring systems of these aryl groups include indanyl, indenyl, tetrahydronaphthalenyl, and tetrahydrobenzoannulenyl.

The term “C₁-C₆ alkyl” refers to a straight or branched chain saturated hydrocarbon containing 1-6 carbon atoms. Examples of a C₁-C₆ alkyl group include, but are not limited to, methyl, ethyl, propyl, butyl, pentyl, isopropyl, isobutyl, sec-butyl and tert-butyl, isopentyl and neopentyl.

The term “carbonyl” as used herein, means a —C(O)— group.

The term “carboxy” as used herein, means a —CO₂H group.

The term “carboxyalkyl” as used herein, means a carboxy group, as defined herein, appended to the parent molecular moiety through an alkyl group, as defined herein. Representative examples of carboxyalkyl include, but are not limited to, carboxymethyl, 2-carboxyethyl, and 3-carboxypropyl.

The term “cyano” as used herein, means a —CN group.

The term “cyanoalkyl” as used herein, means a cyano group, as defined herein, appended to the parent molecular moiety through an alkyl group, as defined herein. Representative examples of cyanoalkyl include, but are not limited to, cyanomethyl, 2-cyanoethyl, and 3-cyanopropyl.

The term “cycloalkenyl” as used herein, means a cyclic hydrocarbon containing from 3 to 8 carbons and containing at least one carbon-carbon double bond formed by the removal of two hydrogens. Representative examples of cycloalkenyl include, but are not limited to, 2-cyclohexen-1-yl, 3-cyclohexen-1-yl, 2,4-cyclohexadien-1-yl and 3-cyclopenten-1-yl.

The term “cycloalkyl” as used herein, means a saturated cyclic hydrocarbon group containing from 3 to 8 carbons, examples of cycloalkyl include cyclopropyl, cyclobutyl, cyclopentyl, cyclohexyl, cycloheptyl, and cyclooctyl.

The term “cycloalkyl groups” of the present invention are optionally substituted with 1, 2, 3, or 4 substituents selected from alkenyl, alkoxy, alkoxyalkoxy, alkoxyalkyl, alkoxycarbonyl, alkyl, alkylcarbonyl, alkylcarbonyloxy, alkylthio, alkynyl, carboxy, cyano, haloalkoxy, haloalkyl, halogen, hydroxy, hydroxyalkyl, mercapto, oxo, —NZ₁Z₂, and (NZ₁Z₂)carbonyl.

The term “cycloalkylene” as used herein,

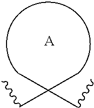

means, wherein A is cycloalkyl or cycloalkyl fused to phenyl.

The term “cycloalkylalkyl” as used herein, means a cycloalkyl group, as defined herein, appended to the parent molecular moiety through an alkyl group, as defined herein. Representative examples of cycloalkylalkyl include, but are not limited to, cyclopropylmethyl, 2-cyclobutylethyl, cyclopentylmethyl, cyclohexylmethyl, 4-cycloheptylbutyl, cyclooctanyl, norboranyl, norborenyl, bicyclo[2.2.2]octanyl, or bicyclo[2.2.2]octenyl.

The term “ethylenedioxy” as used herein, means a —O(CH₂)₂O— group wherein the oxygen atoms of the ethylenedioxy group are attached to the parent molecular moiety through one carbon atom forming a 5 membered ring or the oxygen atoms of the ethylenedioxy group are attached to the parent molecular moiety through two adjacent carbon atoms forming a six membered ring.

The term “formyl” as used herein, means a —C(O)H group.

The term “halogen” or “halo” refers to fluorine (F), chlorine (Cl), bromine (Br) and iodine (I).

The term “haloalkyl” refers to straight or branched saturated hydrocarbon chains containing 1-5 carbon atoms in which at least one of the carbon atoms is substituted with halogen groups such fluorine, chlorine, bromine, iodine. Examples of haloalkyl groups as herein defined include without limitation trifluoromethyl, tribromomethyl, and 1,1,1-trifluoroethyl.

The term “haloalkoxy” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

Unless otherwise specifically defined, the term “heteroaryl,” as used herein, means a monocyclic heteroaryl ring or a bicyclic heteroaryl ring. The monocyclic heteroaryl ring is a 5- or 6-membered ring. The 5-membered ring has two double bonds and contains one, two, three or four heteroatoms independently selected from the group consisting of N, O, and S. The 6-membered ring has three double bonds and contains one, two, three or four heteroatoms independently selected from the group consisting of N, O, and S. The bicyclic heteroaryl ring consists of the 5- or 6-membered heteroaryl ring fused to a phenyl group or the 5- or 6-membered heteroaryl ring fused to a cycloalkyl group or the 5- or 6-membered heteroaryl ring fused to a cycloalkenyl group or the 5- or 6-membered heteroaryl ring fused to another 5- or 6-membered heteroaryl ring. Nitrogen heteroatoms contained within the heteroaryl may be optionally oxidized to the N-oxide or optionally protected with a nitrogen protecting group known to those of skill in the art. The heteroaryl is connected to the parent molecular moiety through any carbon atom or any nitrogen atom contained within the heteroaryl. Representative examples of heteroaryl include, but are not limited to, benzothienyl, benzoxadiazolyl, cirmolinyl, 5,6-dihydroisoquinolinyl, 7,8-dihydroisoquinolinyl, 5,6-dihydroquinolinyl, 7,8-dihydroquinolinyl, furopyridinyl, furyl, imidazolyl, indazolyl, indolyl, isoxazolyl, isoquinolinyl, isothiazolyl, naphthyridinyl, oxadiazolyl, oxazolyl, pyridinyl, pyridazinyl, pyrimidinyl, pyrazinyl, pyrazolyl, pyrrolyl, pyridinium N-oxide, quinolinyl, 5,6,7,8-tetrahydroisoquinolinyl, 5,6,7,8-tetrahydroquinolinyl, tetrazolyl, thiadiazolyl, thiazolyl, thienopyridinyl, thienyl, triazolyl, and triazinyl.

The term “heteroaryl groups” of the present invention are substituted with 0, 1, 2, 3, or 4 substituents independently selected from alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkylthioalkyl, alkynyl, carboxy, carboxyalkyl, cyano, cyanoalkyl, formyl, haloalkoxy, haloalkyl, halogen, hydroxy, hydroxyalkyl, mercapto, nitro, —NZ₁Z₂, and (NZ₁Z₂)carbonyl.

The term “heteroarylalkoxy” as used herein, means a heteroaryl group, as defined herein, appended to the parent molecular moiety through an alkoxy group, as defined herein.

The term “heteroarylalkyl” as used herein, means a heteroaryl, as defined herein, appended to the parent molecular moiety through an alkyl group, as defined herein.

The term “heteroarylalkylthio” as used herein, means a heteroarylalkyl group, as defined herein, appended to the parent molecular moiety through a sulfur atom.

The term “heteroaryloxy” as used herein, means a heteroaryl group, as defined herein, appended to the parent molecular moiety through an oxygen atom.

The term “heteroarylthio” as used herein, means a heteroaryl group, as defined herein, appended to the parent molecular moiety through a sulfur atom.

The term “heterocycle” or “heterocyclic” as used herein, means a monocyclic heterocyclic ring or a bicyclic heterocyclic ring. The monocyclic heterocyclic ring consists of a 3, 4, 5, 6 or 7 membered ring containing at least one heteroatom independently selected from the group consisting of O, N, and S. The 3- or 4-membered ring contains 1 heteroatom selected from the group consisting of O, N and S. The 5-membered ring contains zero or one double bond and one, two or three heteroatoms selected from the group consisting of O, N and S. The 6- or 7-membered ring contains zero, one or two double bonds and one, two or three heteroatoms selected from the group consisting of O, N and S. Representative examples of the monocyclic heterocyclic ring include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocyclic ring consists of the monocyclic heterocyclic ring fused to a phenyl group the monocyclic heterocyclic ring fused to a cycloalkyl group or the monocyclic heterocyclic ring fused to a cycloalkenyl group or the monocyclic heterocyclic ring fused to another monocyclic heterocyclic ring. Representative examples of the bicyclic heterocyclic ring include, but are not limited to, 1,3-benzodioxolyl, 1,3-benzodithiolyl, 2,3-dihydro-1,4-benzodioxinyl, 2,3-dihydro-1-benzofuranyl, 2,3-dihydro-1-behzothienyl, 2,3-dihydro-1Hindolyl, and 1,2,3,4-tetrahydroquinolinyl.

The term “heterocycles” of this invention are substituted with 0, 1, 2, or 3 substituents independently selected from alkenyl, alkoxy, alkoxyalkoxy, alkoxyalkyl, alkoxycarbonyl, alkyl, alkylcarbonyl, alkylcarbonyloxy, alkylthio, alkynyl, carboxy, cyano, cycloalkyl, cycloalkylalkyl, formyl, haloalkoxy, haloalkyl, halogen, hydroxy, hydroxyalkyl, mercapto, oxo, —NZ₁Z₂, and (NZ₁Z₂)carbonyl.

The term “heterocyclealkoxy” as used herein, means a heterocycle group, as defined herein, appended to the parent molecular moiety through an alkoxy group, as defined herein.

The term “heterocyclealkyl” as used herein, means a heterocycle, as defined herein, appended to the parent molecular moiety through an alkyl group, as defined herein.

The term “heterocyclealkylthio” as used herein, means a heterocyclealkyl group, as defined herein, appended to the parent molecular moiety through a sulfur atom. Representative examples of heterocyclealkylthio include, but are not limited to, 2-pyridin-3-ylethythio, 3-quinolin-3-ylpropythio, and 5-pyridin-4-ylpentylthio.

The term “heterocycleoxy” as used herein, means a heterocycle group, as defined herein, appended to the parent molecular moiety through an oxygen atom. Representative examples of heterocycleoxy include, but are not limited to, pyridin-3-yloxy and quinolin-3-yloxy.

The term “heterocyclethio” as used herein, means a heterocycle group, as defined herein, appended to the parent molecular moiety through a sulfur atom.

The term “hydroxy” as used herein, means an —OH group.

The term “hydroxyalkyl” as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “mercapto” as used herein, means a —SH group.

The term “methylenedioxy” as used herein, means a —OCH₂O— group wherein the oxygen atoms of the methylenedioxy are attached to the parent molecular moiety through two adjacent carbon atoms.

The term “nitro” as used herein, means a —NO₂ group.

The term “NZ₁Z₂” as used herein, means two groups, Z₁ and Z₂, which are appended to the parent molecular moiety through a nitrogen atom. Z₁ and Z₂ are each independently selected from the group consisting of hydrogen, alkyl, alkylcarbonyl, and formyl. Representative examples of NZ₁Z₂ include, but are not limited to, amino, methylamino, acetylamino, and acetylmethylamino.

The term “(NZ₁Z₂)carbonyl” as used herein, means a NZ₁Z₂ group, as defined herein, appended to the parent molecular moiety through a carbonyl group, as defined herein. Representative examples of (NZ₁Z₂)carbonyl include, but are not limited to, aminocarbonyl, (methylamino)carbonyl, (dimethylamino)carbonyl, and (ethylmethylamino)carbonyl.

The term “oxo” as used herein, means a ═O moiety.

The term “spiroheterocycle” as used herein, means

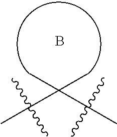

wherein B is a heterocycle or heterocycle fused to phenyl.

The term “sulfonyl” as used herein, means a —SO₂— group.

### DETAILED DESCRIPTION OF THE INVENTION

The invention is based in part on the discovery of novel neuroactive steroids and pharmaceutical compositions thereof, as well as methods of their preparation and use, in therapy of various diseases and conditions.

In one aspect, the invention generally relates to a compound having the structural formula (I):

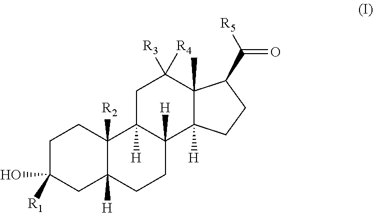

wherein

R₁ is H or a substituted or unsubstituted C₁-C₆ alkyl;

R₂ is H or a substituted or unsubstituted C₁-C₆ alkyl;

each of R₃ and R₄ is independently selected from the group consisting of H, halogen, a substituted or unsubstituted C₁-C₆ alkyl, optionally R₃ and R₄, along with the carbon to which they are attached, may form an exocyclic double bond, or a C₃-C₁₈-membered ring optionally substituted with one or more substituents selected from the group consisting of halogen, OH, CN, C₁-C₅ alkyl, and O—C₁-C₅ alkyl;

R₅ is OR′ or a C₁-C₆ alkyl optionally substituted with heterocyclic or heterobicyclic group, which is optionally substituted with one or more of CN, OH, halogen, a substituted or unsubstituted C₁-C₆ alkyl; provided that, if each of R₃ and R₄ is H, R₅ is a C₁ alkyl substituted with a heterocyclic or heterobicyclic group, which is optionally substituted with C═O(NR^{f}R^{g}), C(R^{f})(R^{g})(OR^{h}), or OR^{h}, wherein each of R′, R^{f} and R^{g} is independently selected from the group consisting of H, a substituted or unsubstituted C₁-C₆ alkyl, and R^{h} is (CH₂CH₂O)ₙCH₃ or CH₂O(CH₂CH₂O)ₙCH₃, wherein n is 1, 2, 3, 4 or 5,

or a pharmaceutically acceptable form or an isotope derivative thereof.

In certain embodiments, at least one of R₃ and R₄ is a halogen.

In certain embodiments, each of R₃ and R₄ is a halogen. In certain embodiments, the halogen is F.

In certain embodiments, each of R₃ and R₄ is H.

In certain embodiments, R₁ is CH₃.

In certain embodiments, R₂ is H.

In certain embodiments, R₅ is a substituted or unsubstituted C₁-C₃ alkyl. In certain embodiments, R₅ is a substituted or unsubstituted methyl. In certain embodiments, R₅ is a methyl substituted with a heterocyclic or heterobicyclic group.

In certain embodiments, the heterocyclic or heterobicyclic group is substituted with one or more of halogen, CN, OH, a substituted or unsubstituted C₁-C₆ alkyl, C═O(NR^{f}R^{g}), C(R^{f})(R^{g})(OR^{h}), or OR^{h}, wherein each of R^{f} and R^{g} is independently selected from the group consisting of H, a substituted or unsubstituted C₁-C₆ alkyl, and R^{h} is (CH₂CH₂O)ₙCH₃ or CH₂O(CH₂CH₂O)ₙCH₃, wherein n is 1, 2, 3, 4 or 5.

In certain embodiments, R₅ is CH₂R^{j}, wherein R^{j} is selected from the group consisting of:

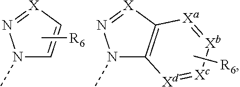

wherein

each of X, X^{a}, X^{b}, X^{c} and X^{d} is independently selected from N and CH; and

R₆ is CN, halogen, C═O(NR^{f}R^{g}), C(R^{f})(R^{g})(OR^{h}), or OR^{h}, wherein each of R^{f} and R^{g} is independently selected from the group consisting of H, a substituted or unsubstituted C₁-C₆ alkyl, and R^{h} is (CH₂CH₂O)ₙCH₃ or CH₂O(CH₂CH₂O)ₙCH₃, wherein n is 1, 2, 3, 4 or 5.

In certain embodiments, X is CH.

In certain embodiments, X is N.

In certain embodiments, each of X^{a}, X^{b}, X^{c} and X^{d} is CH.

In certain embodiments, R₆ is CN.

In certain embodiments, R₆ is F.

In certain embodiments, R₆ is C═O(NH₂).

In certain embodiments, R₆ is O(CH₂CH₂O)ₙCH₃.

In certain embodiments, R₆ is CH₂O(CH₂CH₂O)ₙCH₃.

Exemplary compounds of the invention include:

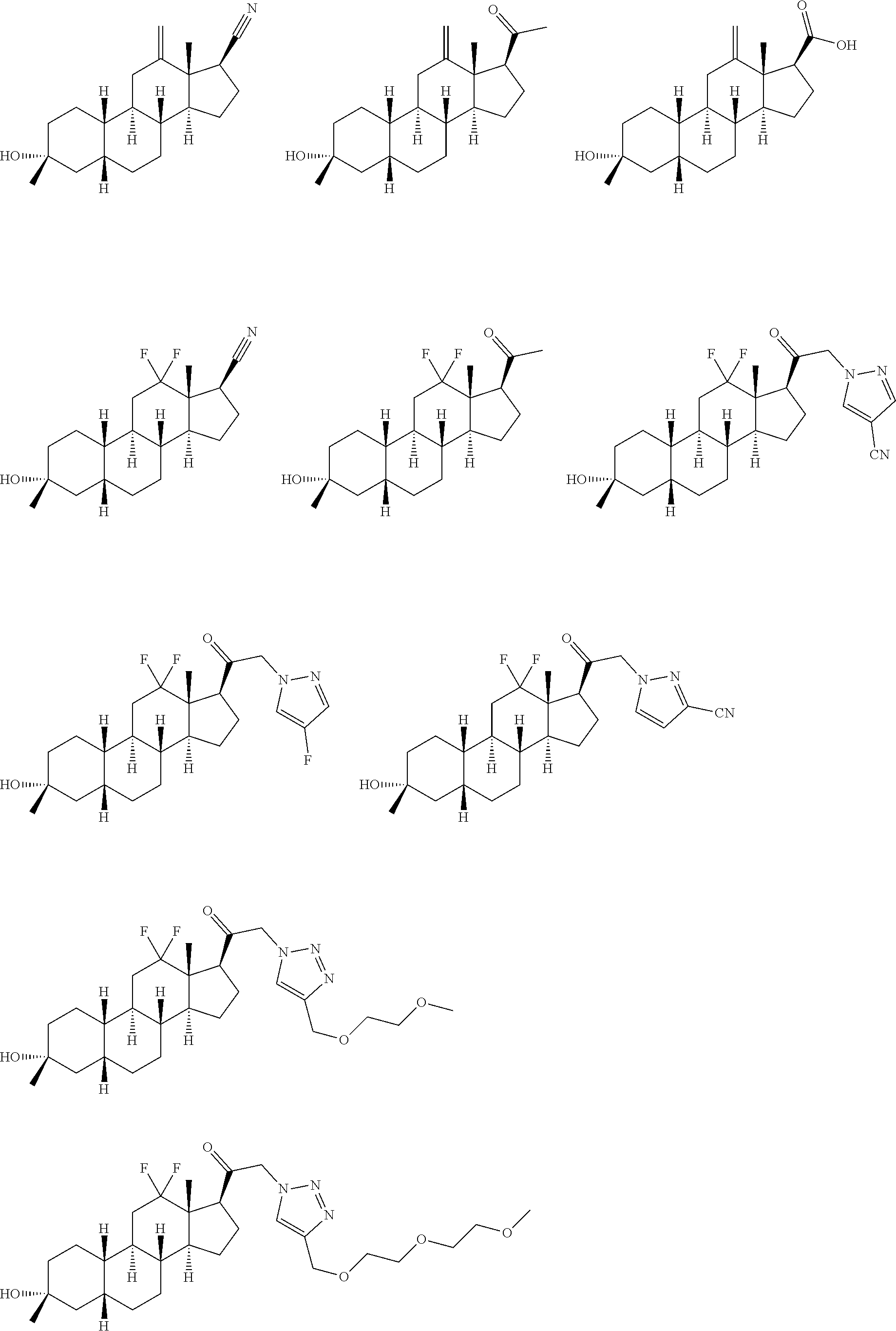

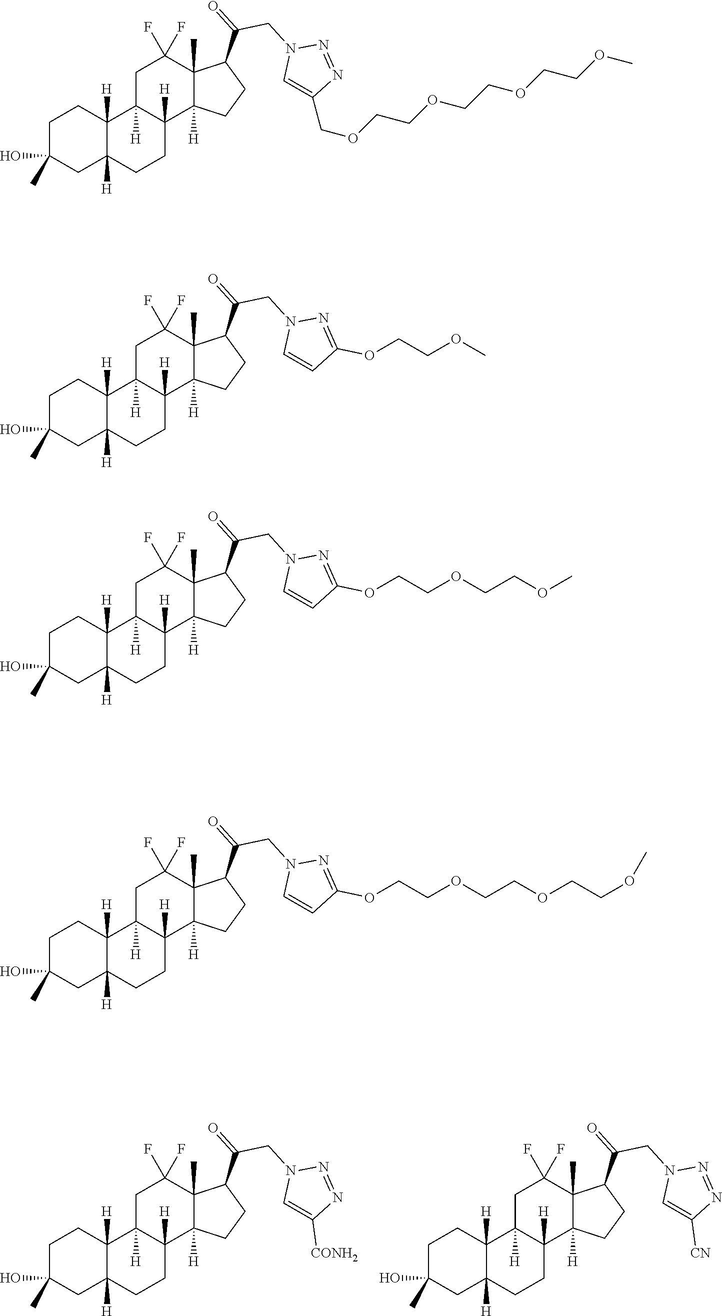

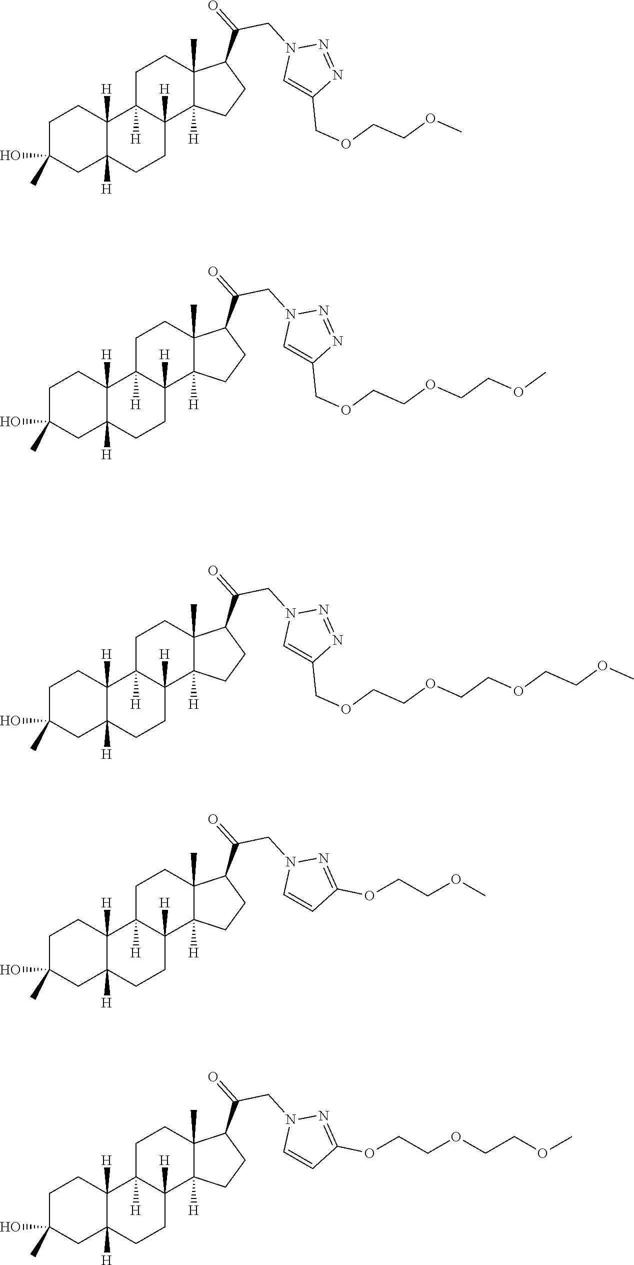

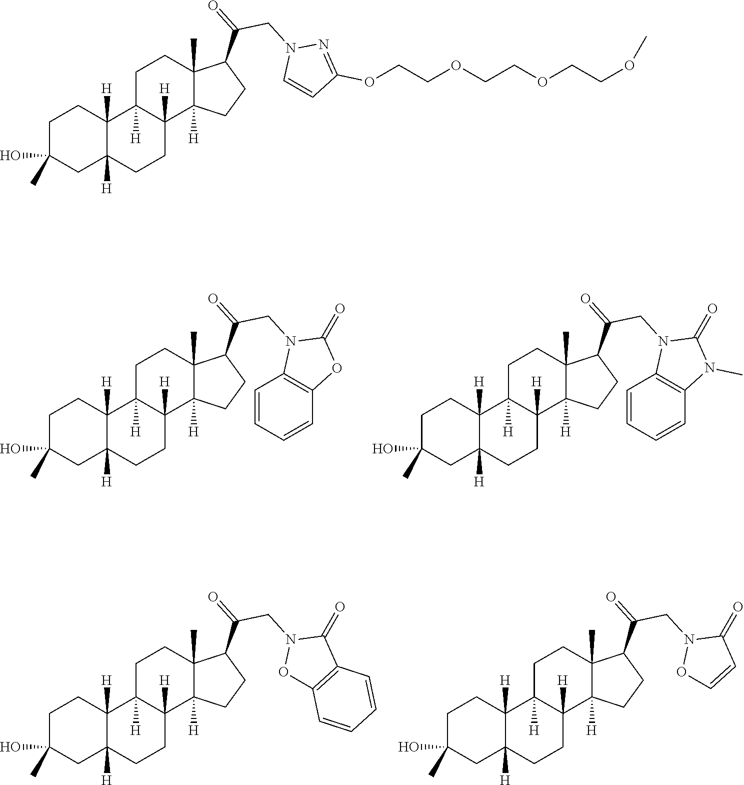

In certain embodiments, the compound has the structural formula:

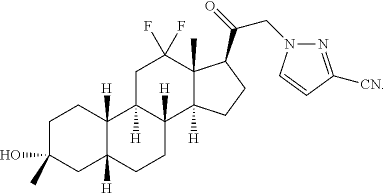

In another aspect, the invention generally relates to a pharmaceutical composition comprising a compound disclosed herein, effective to treat or reduce one or more diseases or disorders, in a mammal, including a human, and a pharmaceutically acceptable excipient, carrier, or diluent.

In another aspect, the invention generally relates to a pharmaceutical composition comprising a compound having the structural formula (I)

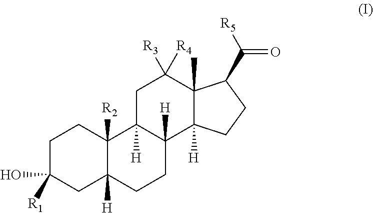

wherein

R₁ is H or a substituted or unsubstituted C₁-C₆ alkyl;

R₂ is H or a substituted or unsubstituted C₁-C₆ alkyl;

each of R₃ and R₄ is independently selected from the group consisting of H, halogen, a substituted or unsubstituted C₁-C₆ alkyl, optionally R₃ and R₄, along with the carbon to which they are attached, may form an exocyclic double bond, or a C₃-C₁₈-membered ring optionally substituted with one or more substituents selected from the group consisting of halogen, OH, CN, C₁-C₅ alkyl, and O—C₁-C₅ alkyl;

R₅ is OR′ or a C₁-C₆ alkyl optionally substituted with heterocyclic or heterobicyclic group, which is optionally substituted with one or more of CN, OH, halogen, a substituted or unsubstituted C₁-C₆ alkyl; provided that, if each of R₃ and R₄ is H, R₅ is a C₁ alkyl substituted with a heterocyclic or heterobicyclic group, which is optionally substituted with C═O(NR^{f}R^{g}), C(R^{f})(R^{g})(OR^{h}), or OR^{h}, wherein each of R′, R^{f} and R^{g} is independently selected from the group consisting of H, a substituted or unsubstituted C₁-C₆ alkyl, and R^{h} is (CH₂CH₂O)ₙCH₃ or CH₂O(CH₂CH₂O)ₙCH₃, wherein n is 1, 2, 3, 4 or 5,

or a pharmaceutically acceptable form or an isotope derivative thereof, effective to treat or reduce one or more diseases or disorders, in a mammal, including a human, and a pharmaceutically acceptable excipient, carrier, or diluent.

In certain embodiments, the pharmaceutical composition of the invention is suitable for oral administration.

In certain embodiments, the pharmaceutical composition of the invention is useful to treat or reduce one or more of PPD, depression, insomnia, sleep apnea, restless legs syndrome, and narcolepsy, emotional disorders, depression, schizophrenia, bipolar disorder, obsessive-compulsive disorder, and other anxiety disorders, behavioral and pharmacological syndrome of dementia, and neurodegenerative diseases.

In certain embodiments, the pharmaceutical composition of the invention is useful to treat or reduce a major depressive disorder.

In certain embodiments, the pharmaceutical composition of the invention is useful to treat or reduce insomnia.

In certain embodiments, the pharmaceutical composition of the invention is useful to treat or reduce a neurodegenerative disease selected from the group consisting of Alzheimer's disease, Parkinson's disease, and seizure disorders, or a related disease or disorder.

In certain embodiments, the pharmaceutical composition of the invention is useful to treat or reduce Alzheimer's disease.

In yet another aspect, the invention generally relates to a unit dosage form comprising a pharmaceutical composition disclosed herein.

In certain embodiments, the unit dosage form is a tablet. In certain embodiments, the unit dosage form is a capsule.

In yet another aspect, the invention generally relates to a method for treating or reducing a disease or disorder, comprising administering to a subject in need thereof a pharmaceutical composition comprising a compound having the structural formula (I):

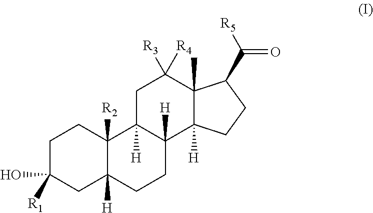

wherein

R₁ is H or a substituted or unsubstituted C₁-C₆ alkyl;

R₂ is H or a substituted or unsubstituted C₁-C₆ alkyl;

each of R₃ and R₄ is independently selected from the group consisting of H, halogen, a substituted or unsubstituted C₁-C₆ alkyl, optionally R₃ and R₄, along with the carbon to which they are attached, may form an exocyclic double bond, or a C₃-C₁₈-membered ring optionally substituted with one or more substituents selected from the group consisting of halogen, OH, CN, C₁-C₅ alkyl, and O—C₁-C₅ alkyl;

R₅ is OR′ or a C₁-C₆ alkyl optionally substituted with heterocyclic or heterobicyclic group, which is optionally substituted with one or more of CN, OH, halogen, a substituted or unsubstituted C₁-C₆ alkyl; provided that, if each of R₃ and R₄ is H, R₅ is a C₁ alkyl substituted with a heterocyclic or heterobicyclic group, which is optionally substituted with C═O(NR^{f}R^{g}), C(R^{f})(R^{g})(OR^{h}), or OR^{h}, wherein each of R′, R^{f} and R^{g} is independently selected from the group consisting of H, a substituted or unsubstituted C₁-C₆ alkyl, and R^{h} is (CH₂CH₂O)ₙCH₃ or CH₂O(CH₂CH₂O)ₙCH₃, wherein n is 1, 2, 3, 4 or 5,

or a pharmaceutically acceptable form or an isotope derivative thereof, effective to treat or reduce one or more of PPD, depression, insomnia, sleep apnea, restless legs syndrome, and narcolepsy, emotional disorders, depression, schizophrenia, bipolar disorder, obsessive-compulsive disorder, and other anxiety disorders, behavioral and pharmacological syndrome of dementia, and neurodegenerative diseases, or a related disease or disorder, in a mammal, including a human.

In yet another aspect, the invention generally relates to a method for treating or reducing a disease or disorder, comprising administering to a subject in need thereof a pharmaceutical composition comprising a compound disclosed herein, wherein the disease or disorder is one or more of PPD, depression, insomnia, sleep apnea, restless legs syndrome, and narcolepsy, emotional disorders, depression, schizophrenia, bipolar disorder, obsessive-compulsive disorder, and other anxiety disorders, behavioral and pharmacological syndrome of dementia, neurodegenerative diseases, or a related disease or disorder.

In certain embodiments, the disease or disorder is a major depressive disorder.

In certain embodiments, the disease or disorder is insomnia.

In certain embodiments, the disease or disorder is a neurodegenerative disease selected from the group consisting of Alzheimer's disease, Parkinson's disease, and seizure disorders, or a related disease or disorder.

In certain embodiments, the disease or disorder is Alzheimer's disease.

In certain embodiments, the method further comprises administering to a subject in need thereof an antipsychotic agent.

In yet another aspect, the invention generally relates to use of a compound disclosed herein, and a pharmaceutically acceptable excipient, carrier, or diluent, in preparation of a medicament for treating a disease or disorder.

In certain embodiments, the disease or disorder is one or more of PPD, depression, insomnia, sleep apnea, restless legs syndrome, and narcolepsy, emotional disorders, depression, schizophrenia, bipolar disorder, obsessive-compulsive disorder, and other anxiety disorders, behavioral and pharmacological syndrome of dementia and neurodegenerative diseases, or a related disease or disorder.

In certain embodiments, the disease or disorder is a major depressive disorder.

In certain embodiments, the disease or disorder is insomnia.

In certain embodiments, the disease or disorder is a neurodegenerative disease selected from the group consisting of Alzheimer's disease, Parkinson's disease, and seizure disorders, or a related disease or disorder.

In certain embodiments, the disease or disorder is Alzheimer's disease.

In certain embodiments, the medicament is for oral administration.

Certain compounds of the present invention may exist in particular geometric or stereoisomeric forms. The present invention contemplates all such compounds, including cis- and transisomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

Isotopically-labeled compounds are also within the scope of the present disclosure. As used herein, an “isotopically-labeled compound” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl respectively.

By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (¹H) with heavier isotopes such as deuterium can afford certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant ¹²C with ¹³C. (See, WO 2007/005643, WO 2007/005644, WO 2007/016361, and WO 2007/016431.)

Stereoisomers (e.g., cis and trans isomers) and all optical isomers of a presently disclosed compound (e.g., R and S enantiomers), as well as racemic, diastereomeric and other mixtures of such isomers are within the scope of the present disclosure.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

Solvates and polymorphs of the compounds of the invention are also contemplated herein. Solvates of the compounds of the present invention include, for example, hydrates.

Any appropriate route of administration can be employed, for example, parenteral, intravenous, subcutaneous, intramuscular, intraventricular, intracorporeal, intraperitoneal, rectal, or oral administration. Most suitable means of administration for a particular patient will depend on the nature and severity of the disease or condition being treated or the nature of the therapy being used and on the nature of the active compound.

Compositions for parenteral injection comprise pharmaceutically-acceptable sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants.

These compositions can also contain adjuvants such as preservative, wetting agents, emulsifying agents, and dispersing agents. Prevention of the action of microorganisms may be ensured by the inclusion of various antibacterial and antifungal agents, for example, paragen, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption, such as aluminum monostearate and gelatin.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically-acceptable and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p. 33 et seq.

Total daily dose of the compositions of the invention to be administered to a human or other mammal host in single or divided doses may be in amounts, for example, from 0.0001 to 300 mg/kg body weight daily and more usually 1 to 300 mg/kg body weight. The dose, from 0.0001 to 300 mg/kg body, may be given twice a day.

Solid dosage forms for oral administration include capsules, tablets, pills, powders, and granules. In such solid dosage forms, the compounds described herein or derivatives thereof are admixed with at least one inert customary excipient (or carrier) such as sodium citrate or dicalcium phosphate or (i) fillers or extenders, as for example, starches, lactose, sucrose, glucose, mannitol, and silicic acid, (ii) binders, as for example, carboxymethylcellulose, alignates, gelatin, polyvinylpyrrolidone, sucrose, and acacia, (iii) humectants, as for example, glycerol, (iv) disintegrating agents, as for example, agar-agar, calcium carbonate, potato or tapioca starch, alginic acid, certain complex silicates, and sodium carbonate, (v) solution retarders, as for example, paraffin, (vi) absorption accelerators, as for example, quaternary ammonium compounds, (vii) wetting agents, as for example, cetyl alcohol, and glycerol monostearate, (viii) adsorbents, as for example, kaolin and bentonite, and (ix) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents. Solid compositions of a similar type may also be employed as fillers in soft and hard-filled gelatin capsules using such excipients as lactose or milk sugar as well as high molecular weight polyethyleneglycols, and the like. Solid dosage forms such as tablets, dragees, capsules, pills, and granules can be prepared with coatings and shells, such as enteric coatings and others known in the art.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like. Besides such inert diluents, the composition can also include additional agents, such as wetting, emulsifying, suspending, sweetening, flavoring, or perfuming agents.

Materials, compositions, and components disclosed herein can be used for, can be used in conjunction with, can be used in preparation for, or are products of the disclosed methods and compositions. It is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

In yet another aspect, the present invention is directed to a method of treating a neurological disease, and abnormal brain function, and/or an emotional disorder in a subject. In some embodiments, the method comprises administering to a subject in need thereof an effective amount of a compound disclosed herein. The method can also comprise administering to the subject in need thereof a pharmaceutical composition of a compound disclosed herein.

In an embodiment, the present disclosure pertains to compounds that selectively modulate the activity of GABA_{A} receptors that contain δ or γ subunits, which encompasses receptors composed of δ or γ subunits in combination with α or β subunits (i.e., α/β/δ or α/β/γ receptors.) The present disclosure also relates to the therapeutic uses of such compounds.

One therapeutic use of a compound of the present invention that modulates the activity of GABA_{A} receptors is to treat patients suffering from PPD or depression. Depression is the prolonged experience of sadness, hopelessness, or worthlessness to a degree that significantly impairs quality of life and the ability to function. MDD is now commonly treated with Selective Serotonin Reuptake Inhibitors (SSRIs) such as Prozac, Zoloft and newer variants, but these agents are of limited effectiveness. Of additional concern is that even when these drugs are effective, the onset of action is may be delayed 4-6 weeks or more, during which time patients are at increased risk of suicide. Consequently, the Food and Drug Administration has inserted a black-box warning on all antidepressants concerning suicide risk. PPD is treated with allopregnanolone infusion and is inconvenient. There is a need for new agents with greater antidepressant efficacy and faster onset of action.

Another therapeutic use of a compound of the present invention that modulates the activity of GABA_{A} receptors is to treat patients suffering from sleeping disorders such as insomnia, sleep apnea, restless legs syndrome, and narcolepsy. Benzodiazepines, Z-drugs, and Orexin Receptor Antagonists have been the mainstay of insomnia treatment, but these medicines have serious side effects such as next-day sleepiness, cognitive impairment, dependency, rebound, and withdrawal. Most of these drugs have long half-lives, leading to next-day sleepiness and post dangers to patients, especially the elderly patients. There is a need for new agents with improved tolerability and faster onset of action.

Yet another therapeutic use of a compound of the present invention that modulates the activity of GABA_{A} receptors is to treat patients suffering from Alzheimer's disease (AD) and behavioral and psychological symptoms of dementia (BPSD). CDC estimated the prevalence of AD and related dementias will double by 2060 from the 5 million patients in 2014. Current treatments only alleviate symptoms and the searching for disease modifying therapeutics in the past two decades, mostly focusing on amyloid homeostasis, has been largely unsuccessful. AD is a complex disease featuring cognitive decline, but there are several comorbidities severely impact a patient's quality of life. Depression is an early comorbidity in mild AD patients, and as the disease progresses anxiety, psychosis, apathy, and other neurological disturbances become more common. It was estimated that BPSD affects up to 90% of the dementia patients over the course of their illness and represent a significant and independent contributor to the overall distress to the patients and their caregivers. Novel approaches are needed to address this public health crisis.

Another therapeutic use for compounds of the present invention is in the treatment of mild cognitive impairment, frontotemporal dementia, multi-infarct dementia, or cognitive dysfunction that occurs after stroke or brain injury.

In one or more embodiments, the abnormal brain function is selected from traumatic brain injury, stroke, and other type of brain injuries. The emotional disorder can be selected from bipolar disorder, obsessive-compulsive disorder, or other anxiety disorders. Other anxiety disorders include general anxiety disorder, social anxiety disorder, phobias and panic disorder.

Another therapeutic use for compounds of the present invention is in the treatment of schizophrenia. Schizophrenia is a debilitating mental disorder encompassing three symptom domains: positive (hallucination, delusions), negative (withdrawal), and cognitive (pervasive reduction in cognitive ability). Schizophrenia typically strikes in early adulthood with the emergence of positive symptoms; however, it is the chronic cognitive deficits that prevent patients from resuming normal activities after the initial onset of symptoms and largely accounts for a lifetime disability.

Given the fundamental role of GABA_{A} receptors in brain function, there are many other therapeutic uses for compounds of the present invention. Such compounds may also be used in the treatment of post-traumatic stress syndrome. Compounds of the present invention may be used to treat individuals suffering from neurological dysfunction, including but not limited to those suffering from AD, Parkinson's disease, Huntington's disease, amyotrophic lateral sclerosis, multiple sclerosis, and seizure disorders. Compounds of the present invention may be used to treat individuals that experience dysfunction caused by abnormal brain development, including but not limited to those suffering from autism and autism spectrum disorders, Fragile X syndrome, tuberous sclerosis, Down's syndrome, Niemann-Pick Type C, and other forms of neurodevelopmental disorders. Such compounds may also be used to treat abnormal brain function that results from infections of the central nervous system, chemical warfare, exposure to toxic agents or other xenobiotics or naturally occurring toxins.

Compounds of the present invention may also be useful in treating pain such as neuropathic pain, pain after nerve or spinal cord injury, pain after tissue damage or burn, or associated pain with diabetes or cardiovascular disease.

The disclosed compounds can be administered in effective amounts to treat or prevent a disorder and/or prevent the development thereof in subjects.

Administration of the disclosed compounds can be accomplished via any mode of administration for therapeutic agents. These modes include systemic or local administration such as oral, nasal, parenteral, transdermal, subcutaneous, vaginal, buccal, rectal or topical administration modes. Additional modes of administration include sublingual, inhalation and intramuscular.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, aerosol, oral dispersible films or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts.

Illustrative pharmaceutical compositions are tablets and gelatin and/or HPMC capsules comprising a Compound of the Invention and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, aliginic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions; using polyalkylene glycols such as propylene glycol, as the carrier.

The disclosed compounds can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564.

Disclosed compounds can also be delivered by the use of monoclonal antibodies as individual carriers to which the disclosed compounds are coupled. The disclosed compounds can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the Disclosed compounds can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, disclosed compounds are not covalently bound to a polymer, e.g., a polycarboxylic acid polymer, or a polyacrylate.

Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

The dosage regimen utilizing the disclosed compound is selected in accordance with a variety of factors including type, species, age, weight, sex and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

In accordance with the foregoing, in a further aspect, the invention relates to a compound of the present invention for use as a medicament, e. g. for the treatment or prevention of a neurological disease, abnormal brain function or an emotional disorder in which modulation of GABA receptors play a role. In a further embodiment, the invention relates to a compound of the present invention for use in the treatment of a disease or disorder mediated by positive allosteric modulation or activation of GABA receptors. In a further embodiment, the disease or disorder is postpartum depression, major depressive disorder, refractory and/or treatment resistant depression. In another embodiment the disease or disorder is a sleeping disorder such as insomnia, sleep apnea, restless legs syndrome, and narcolepsy. In another embodiment the disease or disorder is Alzheimer's disease (AD) or psychological symptoms of dementia. In another embodiment the disease or disorder is bipolar disease. In another embodiment the disease or disorder is post-traumatic stress disorder. In another embodiment the disease or disorder is depression associated with a neurodegenerative disease, such as Parkinson's disease (PD) or Alzheimer's disease (AD). In another embodiment the disease or disorder is neuropathic pain, fibromyalgia, peripheral neuropathy, or seizure disorders.

In a further aspect, the invention relates to the use of a compound of the present invention as an active pharmaceutical ingredient in a medicament, e. g. for the treatment or prevention of a neurological disease, abnormal brain function or an emotional disorder in which modulation of GABA receptors play a role. In a further embodiment, the invention relates to a compound of the present invention for use in the treatment of a disease or disorder mediated by positive allosteric modulation or activation of GABA receptors. In a further embodiment, the disease or disorder is postpartum depression, major depressive disorder, refractory and/or treatment resistant depression. In another embodiment the disease or disorder is a sleeping disorder such as insomnia, sleep apnea, restless legs syndrome, and narcolepsy. In another embodiment the disease or disorder is Alzheimer's disease (AD) or psychological symptoms of dementia. In another embodiment the disease or disorder is bipolar disease. In another embodiment the disease or disorder is post-traumatic stress disorder. In another embodiment the disease or disorder is depression associated with a neurodegenerative disease, such as Parkinson's disease (PD) or Alzheimer's disease (AD). In another embodiment the disease or disorder is neuropathic pain, fibromyalgia, peripheral neuropathy, or seizure disorders.

In a further aspect, the invention relates to the use of a compound of the present invention for the manufacture of a medicament for the treatment or prevention of a neurological disease, abnormal brain function or an emotional disorder in which modulation of GABA receptors play a role. In a further embodiment, the invention relates to a compound of the present invention for use in the treatment of a disease or disorder mediated by positive allosteric modulation or activation of GABA receptors. In a further embodiment, the disease or disorder is postpartum depression, major depressive disorder, refractory and/or treatment resistant depression. In another embodiment the disease or disorder is a sleeping disorder such as insomnia, sleep apnea, restless legs syndrome, and narcolepsy. In another embodiment the disease or disorder is Alzheimer's disease (AD) or psychological symptoms of dementia. In another embodiment the disease or disorder is bipolar disease. In another embodiment the disease or disorder is post-traumatic stress disorder. In another embodiment the disease or disorder is depression associated with a neurodegenerative disease, such as Parkinson's disease (PD) or Alzheimer's disease (AD). In another embodiment the disease or disorder is neuropathic pain, fibromyalgia, peripheral neuropathy, or seizure disorders.

In a further aspect, the invention relates to a method for the treatment or prevention of a neurological disease, abnormal brain function or an emotional disorder in which modulation of GABA receptors play a role. In a further embodiment, the invention relates to a compound of the present invention for use in the treatment of a disease or disorder mediated by positive allosteric modulation or activation of GABA receptors. In a further embodiment, the disease or disorder is postpartum depression, major depressive disorder, refractory and/or treatment resistant depression. In another embodiment the disease or disorder is a sleeping disorder such as insomnia, sleep apnea, restless legs syndrome, and narcolepsy. In another embodiment the disease or disorder is Alzheimer's disease (AD) or psychological symptoms of dementia. In another embodiment the disease or disorder is bipolar disease. In another embodiment the disease or disorder is post-traumatic stress disorder. In another embodiment the disease or disorder is depression associated with a neurodegenerative disease, such as Parkinson's disease (PD) or Alzheimer's disease (AD). In another embodiment the disease or disorder is neuropathic pain, fibromyalgia, peripheral neuropathy, or seizure disorders.

A compound of the present invention can be administered as sole active pharmaceutical ingredient or as a combination with at least one other active pharmaceutical ingredient effective, e. g., in the treatment or prevention of a neurological disease, abnormal brain function or an emotional disorder in which modulation of GABA receptors play a role. Such a pharmaceutical combination may be in the form of a unit dosage form, which unit dosage form comprises a predetermined quantity of each of the at least two active components in association with at least one pharmaceutically acceptable carrier or diluent. Alternatively, the pharmaceutical combination may be in the form of a package comprising the at least two active components separately, e. g. a pack or dispenser-device adapted for the concomitant or separate administration of the at least two active components, in which these active components are separately arranged. In a further aspect, the invention relates to such pharmaceutical combinations.

In a further aspect, the invention therefore relates to a pharmaceutical combination comprising a therapeutically effective amount of a compound of the present invention and a second drug substance, for simultaneous or sequential administration.

In one embodiment, the invention provides a product comprising a compound of the present invention and at least one other therapeutic agent as a combined preparation for simultaneous, separate or sequential use in therapy. In one embodiment, the therapy is the treatment of a disease or condition in which modulation of GABA receptors play a role.

In one embodiment, the invention provides a pharmaceutical composition comprising a compound of the present invention and another therapeutic agent(s). Optionally, the pharmaceutical composition may comprise a pharmaceutically acceptable excipient, as described above.

In one embodiment, the invention provides a kit comprising two or more separate pharmaceutical compositions, at least one of which contains a compound of the present invention. In one embodiment, the kit comprises means for separately retaining said compositions, such as a container, divided bottle, or divided foil packet. An example of such a kit is a blister pack, as typically used for the packaging of tablets, capsules and the like. The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the present invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent. Accordingly, the invention provides an agent of the invention for use in the treatment of a disease or condition in which modulation of GABA receptors play a role, wherein the medicament is prepared for administration with another therapeutic agent. The invention also provides the use of another therapeutic agent for treating a disease or condition in which modulation of GABA receptors play a role, wherein the medicament is administered with a compound of the present invention.

The invention also provides a compound of the present invention for use in a method of treating a disease or condition in which modulation of GABA receptors play a role, wherein the compound of the present invention is prepared for administration with another therapeutic agent. The invention also provides another therapeutic agent for use in a method of treating a disease or condition in which modulation of GABA receptors play a role, wherein the other therapeutic agent is prepared for administration with a compound of the present invention. The invention also provides a compound of the present invention for use in a method of treating a disease or condition in which modulation of GABA receptors play a role, wherein the compound of the present invention is administered with another therapeutic agent. The invention also provides another therapeutic agent for use in a method of treating a disease or condition in which modulation of GABA receptors play a role, wherein the other therapeutic agent is administered with a compound of the invention.

The invention also provides the use of an agent of the invention for treating a disease or condition in which modulation of GABA receptors play a role, wherein the patient has previously (e.g. within 24 hours) been treated with another therapeutic agent. The invention also provides the use of another therapeutic agent for treating a disease or condition in which modulation of GABA receptors play a role, wherein the patient has previously (e.g. within 24 hours) been treated with a compound of the invention.

In one embodiment, the invention relates to a compound of the present invention in combination with another therapeutic agent wherein the other therapeutic agent is selected from:

(a) lithium;

(b) stimulants, such as amphetamine and dextroamphetamine, (Adderall™) or methylphenidate (Ritalin™);

(c) acetylcholinesterase inhibitors, such as donepezil (Aricept™), rivastigmine (Exelon™) and galantamine (Razadyne™);

(d) antidepressant medications for low mood and irritability, such as citalopram (Celexa™) fluoxetine (Prozac™), paroxeine (Paxil™), sertraline (Zoloft™), trazodone (Desyrel™), and tricyclic antidepressants such as amitriptyline (Elavil™);

(e) anxiolytics for anxiety, restlessness, verbally disruptive behavior and resistance, such as lorazepam (Ativan™) and oxazepam (Serax™);

(f) antipsychotic medications for hallucinations, delusions, aggression, agitation, hostility and uncooperativeness, such as aripiprazole (Abilify™), clozapine (Clozaril™), haloperidol (Haldol™) olanzapine (Zyprexa™), quetiapine (Seroquel™), risperidone (Risperdal™) and ziprasidone (Geodon™);

(g) mood stabilizers, such as carbamazepine (Tegretol™) and divalproex (Depakote™);

(h) pregabalin;

(i) gabapentin (Neurontin™);

(j) dopamine agonists such as L-DOPA, pramipexole (Mirapex™) and ropinerol (Requip™);

(k) analgesics including opiates and non-opiates;

(k) carbidopa;

(l) triptans such as sumatriptan (Imitrex™) and zolmitriptan (Zomig™);

(m) nicotinic alpha-7 agonists;

(n) mGluR5 antagonists;

(o) H3 agonists;

(p) amyloid therapy vaccines; and

(q) chemotherapy agents.

The following examples are meant to be illustrative of the practice of the invention, and not limiting in any way.

### EXAMPLES

Abbreviations which have been used in the descriptions of the examples that follow are: ACN: Acetonitrile; AcOH: Acetic acid; BAST: Bis(2-methoxyethyl)aminosulfur trifluoride; BOC: tert-Butyloxycarbonyl; Bu: butyl; t-Bu: tert-butyl; Bu₃SnH: tributyltin hydride; tBuOH: tert-Butanol; t-BuOK: potassium tert-butoxide; Conc.: Concentrated; DCE: 1,1-Dichloroethane; DCM: Dichloromethane; DIPEA: Diisopropylethylamine; DMF: Dimethylformamide; DMSO: Dimethylsulfoxide; EtOAc: Ethyl acetate; EtOH: Ethanol; Et: ethyl; h, hour; HATU: 1-[Bis(dimethylamino)methylene]-1H-1,2,3-triazolo[4,5-b]pyridinium 3-oxid hexafluorophosphate; LDA: HPLC: high pressure liquid chromatography; Lithium diisopropylamide; LiOH H₂O: lithium hydroxide hydrate; L-VcNa: sodium L-ascorbate; MeOH: Methanol; Me: methyl; MeMgBr: methylmagnesium bromide; MS: mass spectrometry; Na₄EDTA: tetrasodium Ethylenediaminetetraacetate; NBS: N-Bromosuccinimide; NMR: Nuclear magnetic resonance; PCC: pyridinium chlorochromate; Pyr: pyridine; psi: pounds per square inch; Sat: Saturated; r.t.: Room temperature; TEA: triethylamine; TFA: Trifluoroacetic acid; TFAA: Trifluoroacetic acid anhydride; THF: Tetrahydrofuran; TMS: trimethylsilane. TosMIC: toluenesulfonylmethyl isocyanide.

Some of the disclosed compounds can be synthesized according to Schemes 1-4. These synthetic routes are intended as an illustration of and not a limitation upon the scope of the invention as defined in the appended claims.

As described in Scheme 1, the diketo intermediates A, which can be made according to known art (Botella, M. et al. J. Med. Chem. 2015, 58, 3500-3511), can be selectively methylated using organometallic reagents to give alcohols B. Intermediates B can be converted to imines C using 2-aminomethylpyridine catalyzed by p-TsOH. The C-12 in imines C can be selectively oxidized to give alcohols D after aqueous work-up (See, Y. Y. et al. J. Am. Chem. Soc. 2015, 137, 13776-13779), which can be converted to nitriles E under basic conditions using TosMIC. PCC oxidation of E can afford ketones F, which can be converted to intermediates G (R₃, R₄ form substituted olefins under these conditions) under the Wittig olefination conditions. Treating G with organometallic reagents can afford ketones H.

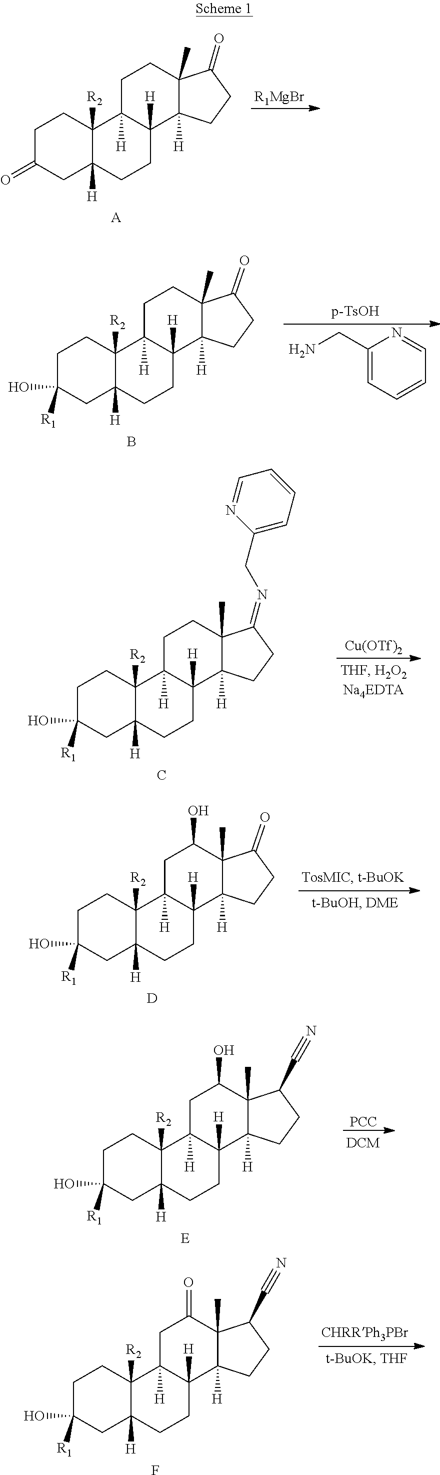

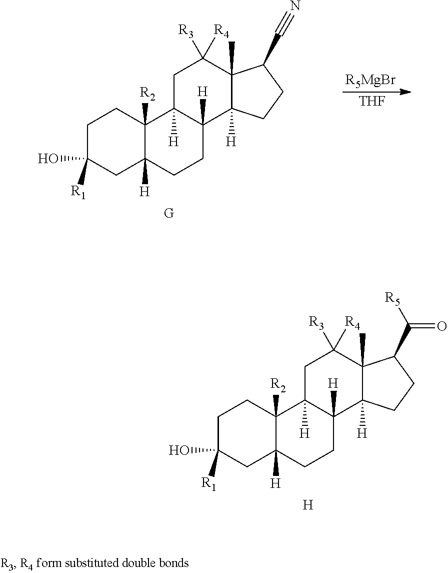

The C12 dihalogenated analogs can be synthesized as described in Scheme 2. The hydroxy group in keto intermediates F can be protected with an acetyl group to give acetates I, which can be dihalogenated using halogenating agents such as BAST to afford intermediates J (R₃, R₄ are halogens under these conditions). Hydrolysis of J can produce acids L and the milder methanolysis of J can afford alcohols K. Conversion of L to the Weinreb amides M under HATU coupling conditions followed by organometallic reagent addition can afford ketone compounds N.

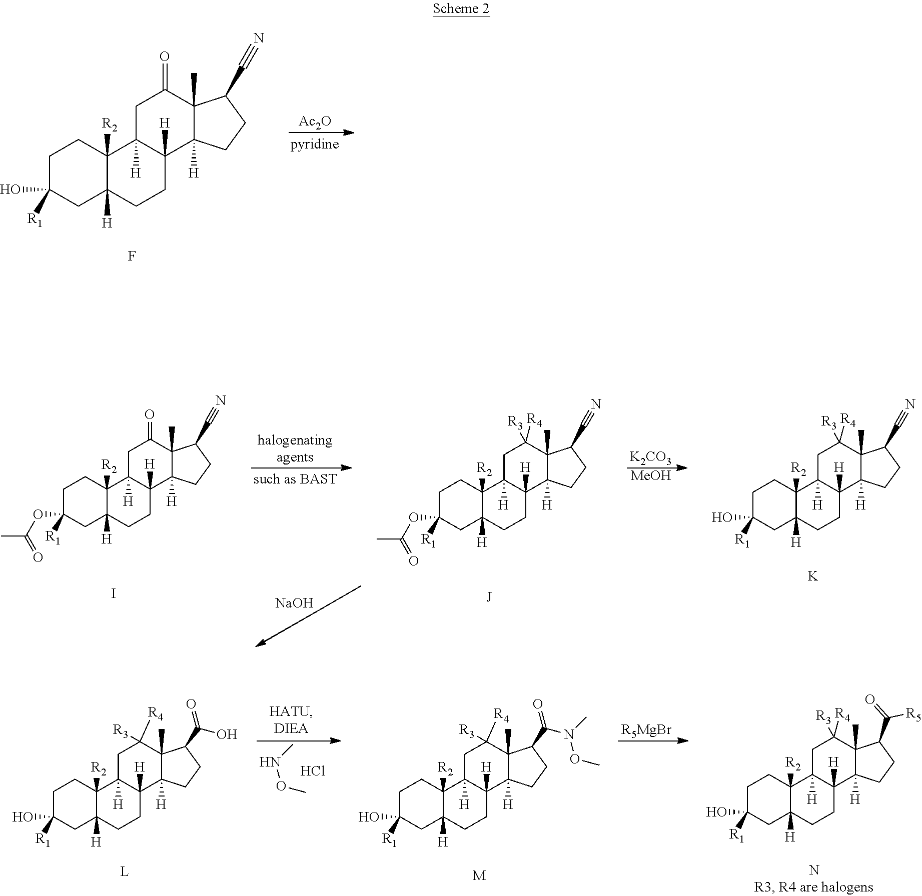

When R₅ is a methyl group in ketones N, the methyl group can be brominated using Br₂ and HBr to give bromo intermediates O, which can be converted to compounds P, Q, R, S, T, U, respectively under basic conditions (Scheme 3).

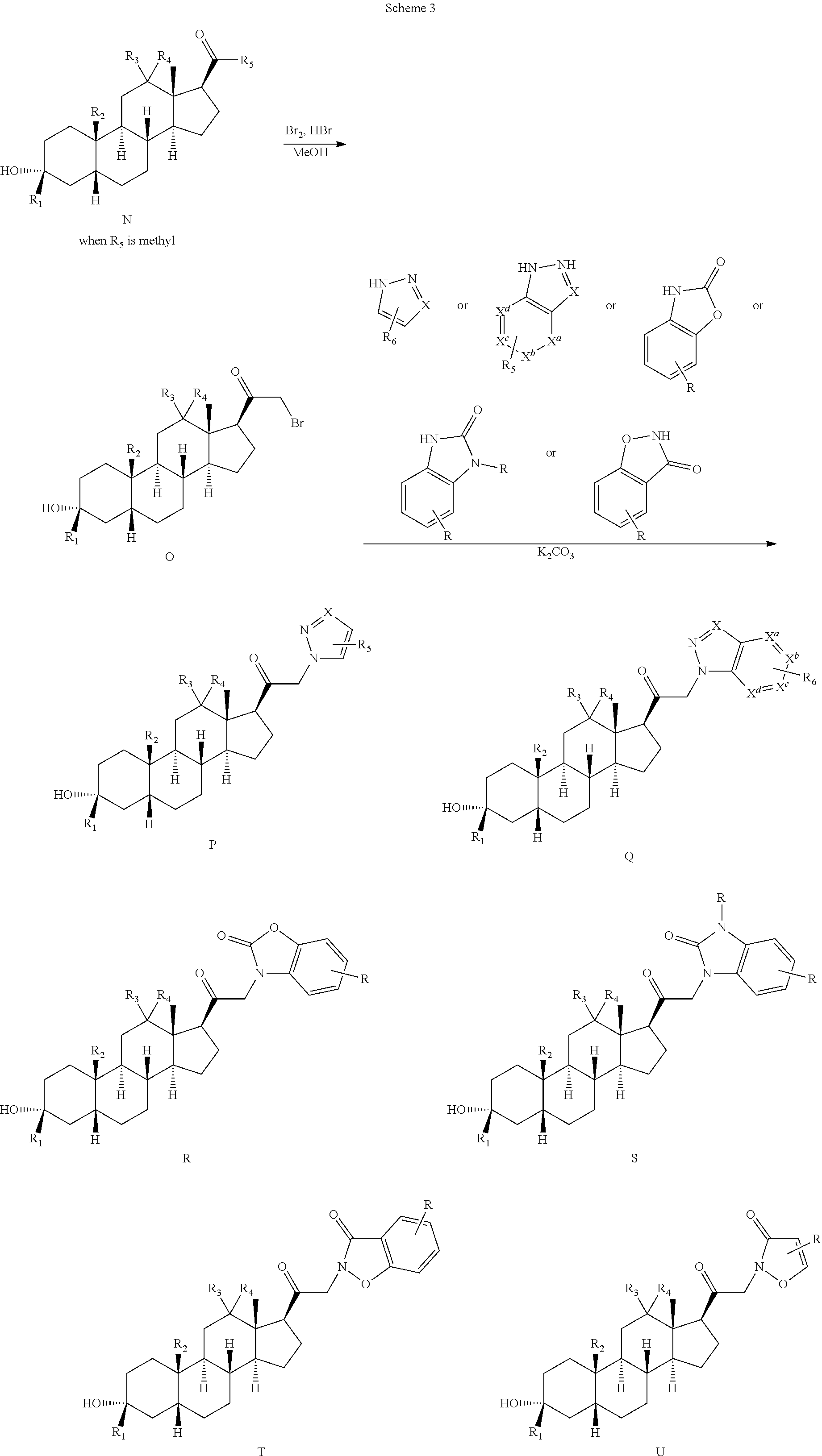

As shown in Scheme 4, bromoketones O can be converted to azido intermediates V using NaN₃, which can react with substituted alkynes catalyzed by CuSO₄ to give triazoles W.

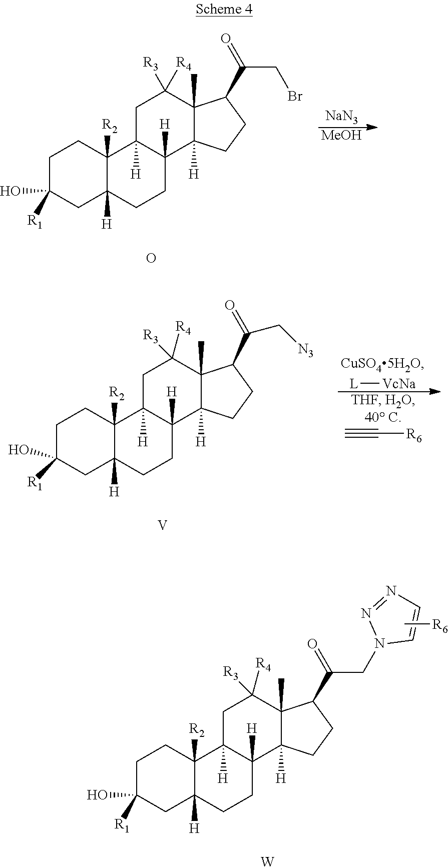

### (3R,5R,8R,9R,10S,13S,14S,17S)-3-hydroxy-3,13-dimethyl-12-methylenehexadecahydro-1H-cyclopenta[a]phenanthrene-17-carbonitrile

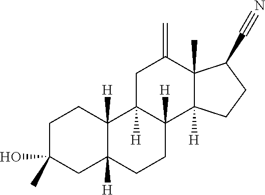

### Example 1

### (5R,8R,9R,10S,13S,14S)-13-methyldodecahydro-1H-cyclopenta[a]phenanthrene-3,17(2H,4H)-dione

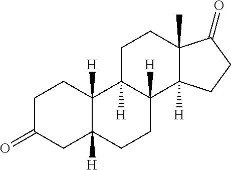

### Example 1-A

A mixture of 10% Pd/C (1.0 g), 4 drops of HBr(aq) and THF (120 mL) was allowed to stir at r.t. under H2 atmosphere for 0.5 h, then a solution of (8R,9S,10R,13S,14S)-13-methyl-7,8,9,10,11,12,13,14,15,16-decahydro-1H-cyclopenta[a]phenanthrene-3,17(2H,6H)-dione (10 g, 36 mmol) in THF (30 mL) was added. The resulting mixture was allowed to stir at r.t. overnight. Additional 1.0 g of 10% Pd/C was added. The reaction mixture was allowed to stir for additional 22 h. The reaction mixture was filtered, the filtrate was evaporated to give the crude product. The crude product was triturated with diethyl ether to give the pure Example 1-A as a white solid (8.0 g, 79%).

### (3R,5R,8R,9R,10S,13 S,14S)-3-hydroxy-3,13-dimethyltetradecahydro-1H-cyclopenta[a]phenanthren-17(2H)-one

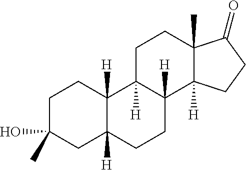

### Example 1-B

To a stirring mixture of dibutylhydroxytoluene (BHT, 9.6 g, 44 mmol) in anhydrous toluene (30 mL) was added AlMe₃ (2M in toluene, 11 mL) at 10° C. under N₂. After stirring at r.t. for 1 h, the reaction mixture was cooled to −78° C., and maintained at the same temperature when a solution of Example 1-A (2.0 g, 7.3 mmol) in toluene (20 mL) was added dropwise under N₂. The mixture was allowed to stir at −78° C. for 1 h, then MeMgBr (3 M in Et₂O, 7.3 mL) was added. The resulting reaction mixture was allowed to stir at −78° C. for 3 h. The reaction mixture was quenched by 10% aqueous citric acid at −78° C. The insoluble materials were filtered off and the filtrate was extracted with EtOAc. The combined organic phases were washed with brine, dried over Na₂SO₄ and concentrated to give the crude product. The crude product was purified by column chromatography (silica gel, eluted with 10-50% EtOAc in heptane) to give Example 1-B (1.8 g, 56%,) as a yellow solid. ¹H NMR (400 MHz, CDCl3) δ 2.43 (dd, 8.7 Hz, 1H), 2.07 (dt, 1H), 1.01-1.95 (m, 24H), 0.86 (s, 3H).

### (3R,5R,8R,9R,10S,13S,14S,Z)-3,13-dimethyl-17-(pyridin-2-ylmethylimino)hexadecahydro-1H-cyclopenta[a]phenanthren-3-ol

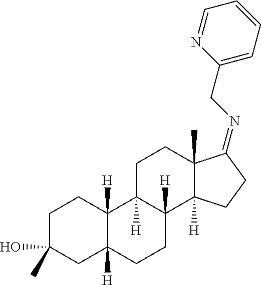

### Example 1-C

A mixture of Example 1-B (20 g, 69 mmol) and 2-aminomethyl pyridine (16 g, 151 mmol) and p-TsOH (1.2 g, 6.9 mmol) in toluene (200 mL) was heated to reflux in a Dean-Stark apparatus until imine formation completed. The reaction mixture was cooled down to r.t., diluted with EtOAc, washed with aqueous NH₄Cl (sat.), aqueous NaHCO₃(sat.) and brine, dried over Na₂SO₄ and concentrated to give the crude product as a brown oil. The crude product was triturated in Et₂O to give Example 1-C (12 g, 62%) as a white solid. ¹H NMR (400 MHz, CDCl3) δ 8.52 (d, 1H), 7.64 (dd, 1H), 7.41 (d, 1H), 7.13 (dd, 1H), 4.76-4.44 (m, 2H), 2.52-2.36 (m, 1H), 2.27 (dt, 1H), 1.10-2.02 (m, 24H), 0.90 (s, 3H).

### (3R,5R,8R,9R,10S,12R,13R,14S)-3,12-dihydroxy-3,13-dimethyltetradecahydro-1H-cyclopenta[a]phenanthren-17(2H)-one

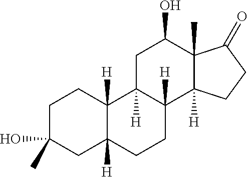

### Example 1-D

A mixture of Example 1-C (11.7 g, 30.7 mmol) and Cu(OTf)₂ (12.2 g, 33.8 mmol) in THF (120 mL) was allowed to stir vigorously at r.t. for 0.5 h, then aqueous H₂O₂ (30%, 17 g, 153 mmol) was added dropwise. The addition of aqueous H₂O₂ was accompanied by a slight exotherm and formation of gas. After the addition, the reaction mixture was allowed to stir at r.t. overnight. 100 mL of EtOAc and 300 mL of aqueous Na₄EDTA (sat.) were added. The aqueous phase was extracted with EtOAc. The combined organic phases were washed with brine, dried over Na₂SO₄, concentrated, and purified by flash column chromatography (silica gel, eluted with 25-50% EtOAC in heptane) to give Example 1-D (6.1 g, 65%) as white solid. ¹H NMR (400 MHz, CDCl3) δ 3.78 (dd, 1H), 2.97 (s, 1H), 2.45 (dd, 1H), 2.18-2.04 (m, 1H), 1.06-2.00 (m, 23H), 0.94 (s, 3H).

### (3R,5R,8R,9R,10S,12R,13S,14S,17S)-3,12-dihydroxy-3,13-dimethylhexadecahydro-1H-cyclopenta[a]phenanthrene-17-carbonitrile

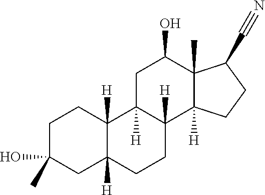

### Example 1-E

To an oven-dried flask was added t-BuOH (110 mL) and t-BuOK (20.1 g, 180 mmol) under N₂. A solution of Example 1-D (5.5 g, 18 mmol) in anhydrous DME (100 mL) was added with stirring. After 30 min, a solution of toluenesulfonylmethyl isocyanide (TosMic, 7 g, 36 mmol) in anhydrous DME (100 mL) was added. The mixture became yellow. The mixture was allowed to stir at r.t. overnight and quenched with water. The mixture was extracted with EtOAc (2×300 mL). The combined organic layer was washed with brine, dried with Na₂SO₄ and concentrated. The residue was purified by flash column chromatography (silica gel, eluted with 30-70% EtOAc in heptance) to give Example 1-E (4.2 g, 74%) as a solid.

### (3R,5R,8R,9R,10S,13S,14S,17S)-3-hydroxy-3,13-dimethyl-12-oxohexadecahydro-1H-cyclopenta[a]phenanthrene-17-carbonitrile

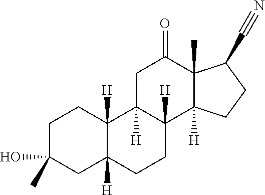

### Example 1-F

To a solution of Example 1-E (3 g, 9.5 mmol) in DCM (200 mL) was added silica gel (15 g) and pyridinium chlorochromate (PCC, 8.1 g, 38 mmol) at r.t. The reaction mixture was allowed to stir at r.t. overnight. The mixture was concentrated and purified by silica gel column chromatography (30-50% EtOAc in heptane) to give Example 1-F (2.5 g, 84%) as a white solid. ¹H NMR (400 MHz, CDCl3) δ 2.95 (t, 1H), 2.38-2.43 (m, 1H), 2.15-2.29 (m, 1H), 1.87-2.01 (m, 3H), 1.68-1.78 (m, 4H), 1.17-1.64 (m, 12H), 1.28 (s, 6H).

### (3R,5R,8R,9R,10S,13S,14S,17S)-3-hydroxy-3,13-dimethyl-12-methylenehexadecahydro-1H-cyclopenta[a]phenanthrene-17-carbonitrile

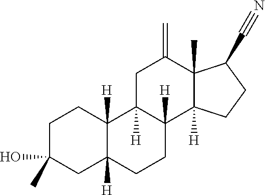

### Example 1

To a stirred suspension of methyl triphenylphosphonium bromide (3.4 g, 9.5 mmol) in anhydrous THF (20 mL) was added t-BuOK (1.1 g, 9.5 mmol) at r.t. under N₂. After stirring at r.t. for 0.5 h, a solution of Example 1-F (1.0 g, 3.2 mmol) in THF (10 mL) was added. The resulting reaction mixture was allowed to stir at r.t. overnight. The reaction mixture was quenched by aqueous NH₄Cl (sat.) and extracted with EtOAc. The combined organic phases were washed with brine, dried over Na₂SO₄, concentrated, and purified by silica gel column chromatography (eluted with 20-50% EtOAc in heptane) to give Example 1 (0.9 g, 91%) as a white solid. ¹H NMR (400 MHz, CDCl3) δ 4.74-4.72 (m, 2H), 2.71 (t, 1H), 2.29 (dd, 1H), 2.25-2.15 (m, 1H), 2.04-1.62 (m, 7H), 1.51-1.22 (m, 13H), 1.27 (s, 3H), 1.07 (s, 3H).

### 1-((3R,5R,8R,9R,10S,13S,14S,17S)-3-hydroxy-3,13-dimethyl-12-methylenehexadecahydro-1H-cyclopenta[a]phenanthren-17-yl)ethan-1-one

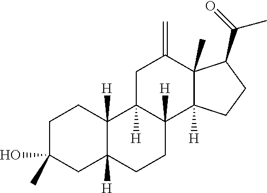

### Example 2

To a stirring solution of Example 1 (0.56 g, 1.8 mmol) in anhydrous THF (40 mL) was added MeMgBr (3M in diethyl ether, 18 mL, 54 mmol) at r.t. under N₂. The reaction mixture was allowed to stir at reflux overnight. The reaction mixture was cooled to r.t., poured into ice-cold aqueous NH₄Cl (sat.), extracted with EtOAc. The combined organic phases were washed with brine, dried over Na₂SO₄ and concentrated to give the crude product as a yellow oil. The crude product was purified by silica gel column chromatography (eluted with 10-20% EtOAc in heptane) to provide Example 2 (0.46 g, 80.5%) as a white solid. ¹H NMR (400 MHz, CDCl3) δ 4.66 (m, 2H), 2.95 (t, 1H), 2.23 (dd, 1H), 2.18 (s, 3H), 2.04-1.62 (m, 7H), 1.49-1.01 (m, 14H), 1.27 (s, 3H), 0.92 (s, 3H).

### (3R,5R,8R,9R,10S,13S,14S,17S)-12,12-difluoro-3-hydroxy-3,13-dimethylhexadecahydro-1H-cyclopenta[a]phenanthrene-17-carboxylic Acid

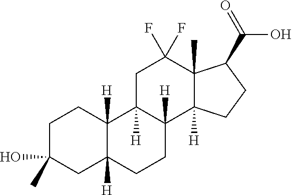

### Example 3

### (3R,5R,8R,9R,10S,13 S,14S,17S)-17-cyano-3,13-dimethyl-12-oxohexadecahydro-1H-cyclopenta[a]phenanthren-3-yl Acetate

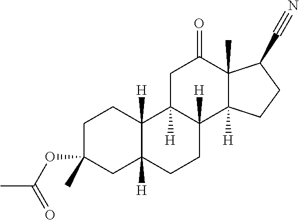

### Example 3-A

To a solution of Example 1-F (4.2 g, 13 mmol) in Ac₂O (20 mL) was added anhydrous pyridine (20 mL), the reaction mixture was allowed to stir at 90° C. overnight. The mixture was concentrated under vacuum to remove most Ac₂O and pyridine, then aqueous citric acid (30 mL) was added. The mixture was extracted with EtOAc (3×30 mL). The combined organic layer was washed successively with aqueous citric acid (3×20 mL), water (30 mL), aqueous NaHCO₃ (30 mL) and brine (30 mL), dried over Na₂SO₄, and concentrated to give compound Example 3-A. ¹H NMR (400 MHz, CDCl3) δ 2.95 (t, 1H), 2.39-2.43 (m, 1H), 2.13-2.26 (m, 2H), 1.96 (s, 3H), 1.54 (s, 3H), 1.17-2.01 (m, 18H), 1.26 (s, 3H).

### (3R,5R,8R,9R,10S,13 S,14S,17S)-17-cyano-12,12-difluoro-3,13-dimethylhexadecahydro-1H-cyclopenta[a]phenanthren-3-yl Acetate

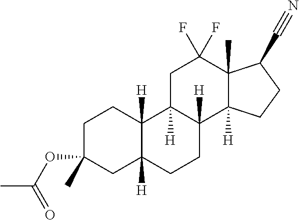

### Example 3-B

To a solution of Example 3-A (crude, 13 mmol) in anhydrous DCE (110 mL) was added BAST (24 mL, 0.13 mol), the reaction mixture was allowed to stir at 90° C. overnight. The mixture was cooled to r.t. and poured into ice water (50 mL). The mixture was extracted with EtOAc (60 mL×3). The combined organic phases were washed successively with aqueous NaHCO₃, water, 2N HCl(aq), and brine. The mixture was dried over Na₂SO₄ and purified by flash column chromatography (silica gel, eluted with PE/EtOAc=20:1) to give Example 3-B (3.7 g, 74% over 2 steps). ¹H NMR (400 MHz, CDCl3) δ 3.03 (t, 1H), 2.04-2.29 (m, 2H), 1.98 (s, 3H), 1.54 (s, 3H), 1.17-2.01 (m, 19H), 1.13 (s, 3H).

### (3R,5R,8R,9R,10S,13S,14S,17S)-12,12-difluoro-3-hydroxy-3,13-dimethylhexadecahydro-1H-cyclopenta[a]phenanthrene-17-carboxylic Acid

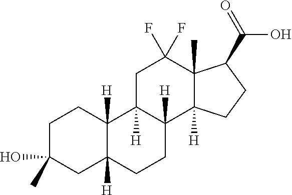

### Example 3

To a solution of Example 3-B (5.2 g, 14 mmol) in ethylene glycol (100 mL) was added NaOH (16 g, 0.41 mol). The reaction mixture was stirred at 160° C. for overnight. After cooling to r.t., water (150 mL) was added, and the mixture adjusted to pH 2 with conc. HCl (˜37 mL). The precipitates were collected by filtration and washed with water to give Example 3 (1.4 g, yield: 88%) as white solid. ¹H NMR (400 MHz, CDCl3) δ 3.15 (t, 1H), 0.97-2.24 (m, 21H), 1.29 (s, 3H), 0.99 (s, 3H).

### (3R,5R,8R,9R,10S,13S,14S,17S)-12,12-difluoro-3-hydroxy-3,13-dimethylhexadecahydro-1H-cyclopenta[a]phenanthrene-17-carbonitrile

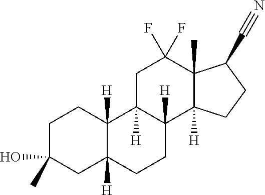

### Example 4

To a solution of Example 3-B (76 mg, 0.2 mmol) in MeOH (10 mL) was added K₂CO₃ (83 mg, 0.6 mmol) and the reaction mixture was allowed to stir at r.t. overnight. The mixture was concentrated to remove MeOH, then EtOAc (20 mL) and water (20 mL) were added. The organic layer was washed with water and brine, dried with Na₂SO₄, and concentrated to give Example 4 (60 mg, 88.7%). ¹H NMR (400 MHz, CDCl3) δ 3.05 (t, 1H), 2.08-2.29 (m, 2H), 1.54 (s, 3H), 1.26-2.04 (m, 19H), 1.11 (s, 3H).

### 1-((3R,5R,8R,9R,10S,13S,14S,17S)-12,12-difluoro-3-hydroxy-3,13-dimethylhexadecahydro-1H-cyclopenta[a]phenanthren-17-yl)ethan-1-one

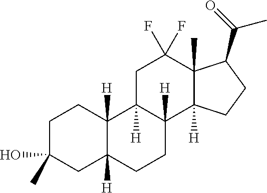

### Example 5

### (3R,5R,8R,9R,10S,13S,14S,17S)-12,12-difluoro-3-hydroxy-N-methoxy-N,3,13-trimethylhexadecahydro-1H-cyclopenta[a]phenanthrene-17-carboxamide

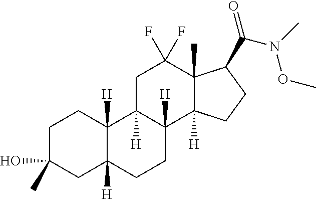

### Example 5-A

To a solution of Example 3 (1.1 g, 3 mmol) in DMF (25 mL) was added N,O-dimethylhydroxylamine hydrochloride (439 mg, 4.5 mmol), HATU (1.7 g, 4.5 mmol), diisopropylethylamine (DIPEA, 1.49 mL, 9 mmol). The reaction mixture was allowed to stir at r.t. overnight. Water (100 mL) was added, and the mixture was extracted with EtOAc (3×30 mL). The combined organic layer was washed successively with 0.1 N aqueous HCl, water and brine, then dried over Na₂SO₄. The solvent was removed under vacuum, and the residue was purified by flash column chromatography (silica gel, eluted with 15-40% EtOAc in heptane) to give Example 5-A (800 mg, 67%). ¹HNMR (400 MHz, CDCl3) δ 3.63 (s, 3H), 3.45 (m, 1H), 3.15 (s, 3H), 1.07-2.05 (m, 21H), 1.27 (s, 3H), 1.00 (s, 3H).

### 1-((3R,5R,8R,9R,10S,13S,14S,17S)-12,12-difluoro-3-hydroxy-3,13-dimethylhexadecahydro-1H-cyclopenta[a]phenanthren-17-yl)ethan-1-one

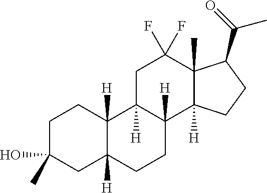

### Example 5

To an oven-dried flask was added anhydrous THF (25 mL) and Example 5-A (800 mg, 2 mmol) under N₂. The solution was cooled to 0° C. and MeMgCl (3N in THF, 7 mL, 20 mmol) was added. The mixture was allowed to stir at r.t. for 1 h, then quenched with aqueous NH₄Cl (100 mL). The mixture was extracted with EtOAc (3×50 mL). The combined organic layer was washed with aqueous NH₄Cl, then brine, dried over Na₂SO₄ and concentrated to give Example 5 (760 mg, 100%) as a solid. ¹H NMR (400 MHz, CDCl3) δ 3.32 (t, 1H), 2.25 (d, 3H), 2.03-2.18 (m, 2H), 1.27 (s, 3H), 1.05-1.86 (m, 19H), 0.79 (s, 3H).

### 1-(2-((3R,5R,8R,9R,10S,13S,14S,17S)-12,12-difluoro-3-hydroxy-3,13-dimethylhexadecahydro-1H-cyclopenta[a]phenanthren-17-yl)-2-oxoethyl)-1H-pyrazole-4-carbonitrile

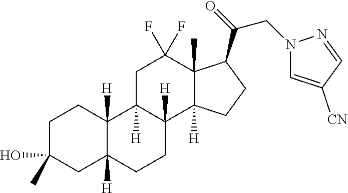

### Example 6

### 2-bromo-1-((3R,5R,8R,9R,10S,13S,14S,17S)-12,12-difluoro-3-hydroxy-3,13-dimethylhexadecahydro-1H-cyclopenta[a]phenanthren-17-yl)ethanone

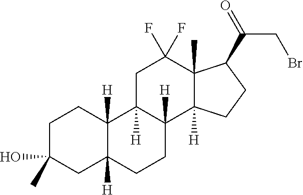

### Example 6-A

To a solution of Example 5 (1.1 g, 3.0 mmol) in MeOH (30 mL) was added HBr (48% in water, 60 μL, 0.45 mmol) and Br₂ (600 mg, 192 μL, 3.7 mmol). The reaction mixture was allowed to stir at room r.t for 1 h. The mixture was concentrated to remove MeOH. The residue was quenched by aqueous NaHCO₃ (40 mL), and extracted with EtOAc (3×30 mL). The combined organic layer was washed successively with aqueous NaHCO₃, water, brine, then dried over Na₂SO₄, filtered, and concentrated to give Example 6-A. ¹H NMR (400 MHz, CDCl3) δ 4.09 (q, 2H), 3.53 (t, 1H), 2.01-2.24 (m, 2H), 1.06-1.87 (m, 19H), 1.21 (s, 3H), 0.75 (s, 3H).

### 1-(2-((3R,5R,8R,9R,10S,13S,14S,17S)-12,12-difluoro-3-hydroxy-3,13-dimethylhexadecahydro-1H-cyclopenta[a]phenanthren-17-yl)-2-oxoethyl)-1H-pyrazole-4-carbonitrile

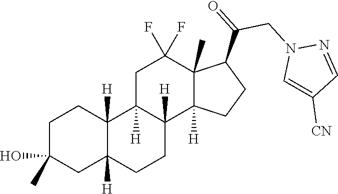

### Example 6

To a mixture of Example 6-A (120 mg, 0.27 mmol) and K₂CO₃ (76 mg, 0.55 mmol) in acetone (10 mL) was added 4-cyanopyrazole (39 mg, 0.42 mmol). The reaction mixture was allowed to stir at r.t. overnight. The reaction mixture was quenched by water (40 mL) and extracted with EtOAc (3×20 mL). The combined organic layer was washed with brine, dried over Na₂SO₄, concentrated, and purified by silica gel column chromatography (30-50% EtOAc in heptane) to give Example 6 (55 mg, 44.7%) as a white solid. ¹H NMR (400 MHz, CDCl3) δ 7.82 (s, 1H), 7.79 (s, 1H), 5.32 (d, 1H), 5.00 (d, 1H), 3.30 (t, 1H), 2.15 (m, 2H), 1.08-1.88 (m, 21H), 1.28 (s, 3H), 0.84 (s, 3H).

### 1-((3R,5R,8R,9R,10S,13S,14S,17S)-12,12-difluoro-3-hydroxy-3,13-dimethylhexadecahydro-1H-cyclopenta[a]phenanthren-17-yl)-2-(4-fluoro-1H-pyrazol-1-yl)ethan-1-one

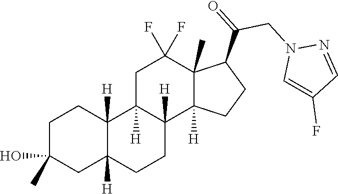

### Example 7

Example 7 was made in the same fashion as Example 6 substituting 4-cyanopyrazole with 4-fluoropyrazole. ¹H NMR (400 MHz, CDCl3) δ 7.38 (d, 1H), 7.25 (d, 1H), 5.16 (d, 1H), 4.84 (d, 1H), 3.27 (t, 1H), 2.23-2.07 (m, 2H), 1.08-1.88 (m, 21H), 1.27 (s, 3H), 0.84 (s, 3H).

### 1-(2-((3R,5R,8R,9R,10S,13S,14S,17S)-12,12-difluoro-3-hydroxy-3,13-dimethylhexadecahydro-1H-cyclopenta[a]phenanthren-17-yl)-2-oxoethyl)-1H-pyrazole-3-carbonitrile

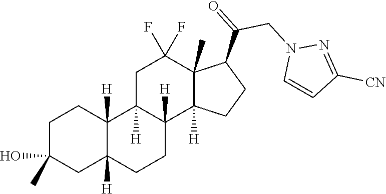

### Example 8

Example 8 was synthesized as for Example 6 substituting 4-cyanopyrazole with 3-cyanopyrazole. ¹H NMR (400 MHz, CDCl3) δ 7.42 (d, J=2.4 Hz, 1H), 6.72 (d, 1H), 5.34 (d, 1H), 5.02 (d, 1H), 3.30 (t, 1H), 2.26-2.08 (m, 2H), 1.09-1.88 (m, 21H), 1.28 (s, 3H), 0.84 (s, 3H).

### 1-((3R,5R,8R,9R,10S,13 S,14S,17S)-12,12-difluoro-3-hydroxy-3,13-dimethylhexadecahydro-1H-cyclopenta[a]phenanthren-17-yl)-2-(4-((2-methoxyethoxy)methyl)-1H-1,2,3-triazol-1-yl)ethan-1-one

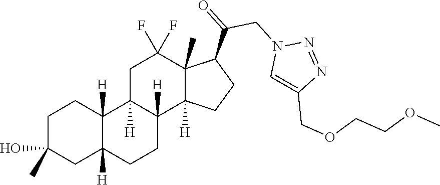

### Example 9

### 3-(2-methoxyethoxy)prop-1-yne

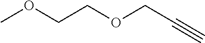

### Example 9-A

To a stirred suspension of NaH (60% in mineral oil, 2.4 g, 60 mmol) in dry THF (20 mL) was added a solution of 2-methoxyethanol (3.8 g, 50 mmol) in dry THF (20 mL) dropwise at 0° C. under N₂. After stirring for 0.5 h, a solution of propargyl bromide (6.5 g, 55 mmol) in anhydrous THF (10 mL) was added. The reaction mixture was allowed to stir at 0° C. for 1 h. The reaction mixture was quenched with water (30 mL), extracted with EtOAc (3×30 mL). The combined organic phases were washed with brine, dried over Na₂SO₄, concentrated, and purified by flash column chromatography (silica gel, eluted with 5-10% EtOAc in heptane) to give Example 9-A (0.8 g, 14%) as a yellow oil. ¹HNMR (400 MHz, CDCl3) δ 4.21 (d, 2H), 3.72-3.65 (m, 2H), 3.62-3.51 (m, 2H), 3.39 (s, 3H), 2.43 (t, 1H).

### 2-azido-1-((3R,5R,8R,9R,10S,13S,14S,17S)-12,12-difluoro-3-hydroxy-3,13-dimethylhexadecahydro-1H-cyclopenta[a]phenanthren-17-yl)ethanone

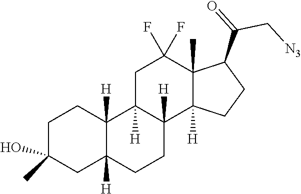

### Example 9-B

A mixture of Example 6-A (510 mg, 1.18 mmol) and sodium azide (153 mg, 2.35 mmol) in MeOH (10 mL) was allowed to stir at r.t. under N₂ overnight. The reaction mixture was poured into water (20 mL), extracted with EtOAc (3×20 mL). The combined organic phases were washed with brine, dried over Na₂SO₄, concentrated, and purified by flash column chromatography (silica gel, eluted with 8-20% EtOAc in heptane) to give Example 9-B (270 mg, 58%) as a white solid. ¹H NMR (400 MHz, CDCl3) δ 4.30 (d, 1H), 3.88 (dd, 1H), 3.22 (t, 1H), 2.26-1.99 (m, 2H), 1.86-1.30 (m, 17H), 1.27 (s, 3H), 1.16-1.06 (m, 2H), 0.84 (s, 3H).

### 1-((3R,5R,8R,9R,10S,13 S,14S,17S)-12,12-difluoro-3-hydroxy-3,13-dimethylhexadecahydro-1H-cyclopenta[a]phenanthren-17-yl)-2-(4-((2-methoxyethoxy)methyl)-1H-1,2,3-triazol-1-yl)ethan-1-one

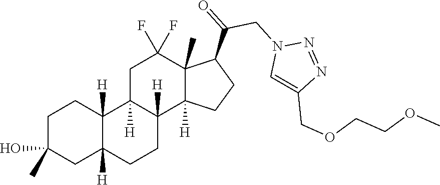

### Example 9

A mixture of Example 9-B (80 mg, 0.2 mmol), Example 9-A (28 mg, 0.24 mmol), CuSO₄.5H₂O (5 mg, 0.02 mmol) and L-Sodium ascorbate (16 mg, 0.08 mmol) in THF/H₂O (4 mL/1 mL) was allowed to stir at 40° C. under N₂ overnight. The reaction mixture was quenched with water (10 mL), extracted with EtOAc (3×10 mL). The combined organic phases were washed with brine, dried over Na₂SO₄, concentrated, and purified by flash column chromatography (silica gel, eluted with 2-10% MeOH in DCM) to give Example 9 (73 mg, 72%) as a white solid. ¹H NMR (400 MHz, CDCl3) δ 7.56 (s, 1H), 5.48 (d, 1H), 5.24 (d, 1H), 4.72 (s, 2H), 3.68 (dd, 2H), 3.55 (dd, 2H), 3.37 (m, 4H), 2.26-2.08 (m, 2H), 1.87-1.31 (m, 17H), 1.27 (s, 3H), 1.17-1.11 (m, 2H), 0.84 (s, 3H).

### 1-((3R,5R,8R,9R,10S,13 S,14S,17S)-12,12-difluoro-3-hydroxy-3,13-dimethylhexadecahydro-1H-cyclopenta[a]phenanthren-17-yl)-2-(4-((2-(2-methoxyethoxy)ethoxy)methyl)-1H-1,2,3-triazol-1-yl)ethan-1-one

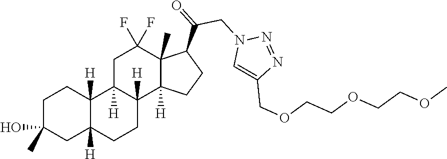

### Example 10

### 3-(2-(2-methoxyethoxy)ethoxy)prop-1-yne

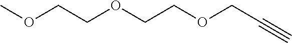

### Example 10-A

Example 10-A was synthesized as for Example 9-A substituting 2-methoxyethanol with 2-(2-methoxyethoxy)ethanol. ¹H NMR (400 MHz, CDCl3) δ 4.21 (d, 2H), 3.75-3.63 (m, 7H), 3.58-3.52 (m, 2H), 3.38 (s, 3H), 2.43 (t, 1H).

### 1-((3R,5R,8R,9R,10S,13 S,14S,17S)-12,12-difluoro-3-hydroxy-3,13-dimethylhexadecahydro-1H-cyclopenta[a]phenanthren-17-yl)-2-(4-((2-(2-methoxyethoxy)ethoxy)methyl)-1H-1,2,3-triazol-1-yl)ethan-1-one

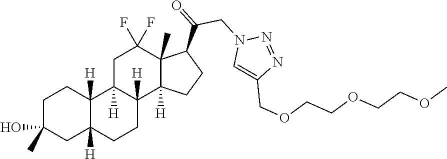

### Example 10

Example 10 was made in the same fashion as Example 9 substituting Example 9-A with Example 10-A. ¹H NMR (400 MHz, CDCl3) δ 7.56 (s, 1H), 5.48 (d, 1H), 5.24 (d, 1H), 4.72 (s, 2H), 3.73-3.61 (m, 6H), 3.55 (dd, 2H), 3.37 (m, 4H), 2.22-2.09 (m, 2H), 1.89-1.31 (m, 17H), 1.28 (s, 3H), 1.18-1.11 (m, 2H), 0.84 (s, 3H).

### 2-(4-(2,5,8,11-tetraoxadodecyl)-1H-1,2,3-triazol-1-yl)-1-((3R,5R,8R,9R,10S,13 S,14S,17S)-12,12-difluoro-3-hydroxy-3,13-dimethylhexadecahydro-1H-cyclopenta[a]phenanthren-17-yl)ethan-1-one

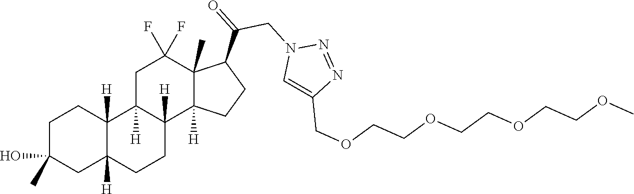

### Example 11

### 2,5,8,11-tetraoxatetradec-13-yne

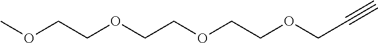

### Example 11-A

Example 11-A was made in the same fashion as Example 9-A substituting 2-methoxyethanol with 2-(2-(2-methoxyethoxy)ethoxy)ethanol. ¹H NMR (400 MHz, CDCl3) δ 4.21 (d, 2H), 3.75-3.63 (m, 10H), 3.58-3.52 (m, 2H), 3.38 (s, 3H), 2.43 (t, 1H).

### 2-(4-(2,5,8,11-tetraoxadodecyl)-1H-1,2,3-triazol-1-yl)-1-((3R,5R,8R,9R,10S,13 S,14S,17S)-12,12-difluoro-3-hydroxy-3,13-dimethylhexadecahydro-1H-cyclopenta[a]phenanthren-17-yl)ethan-1-one

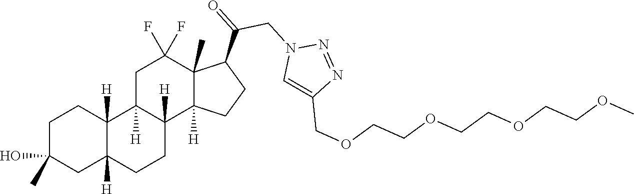

### Example 11

Example 11 was made in the same fashion as Example 9 substituting Example 9-A with Example 11-A. ¹H NMR (400 MHz, CDCl3) δ 7.58 (s, 1H), 5.48 (d, 1H), 5.24 (d, 1H), 4.71 (s, 2H), 3.74-3.60 (m, 10H), 3.57-3.51 (m, 2H), 3.37 (m, 4H), 2.21-2.11 (m, 2H), 1.87-1.31 (m, 17H), 1.28 (s, 3H), 1.19-1.11 (m, 2H), 0.84 (s, 3H).

### 1-((3R,5R,8R,9R,10S,13S,14S,17S)-12,12-difluoro-3-hydroxy-3,13-dimethylhexadecahydro-1H-cyclopenta[a]phenanthren-17-yl)-2-(3-(2-methoxyethoxy)-1H-pyrazol-1-yl)ethan-1-one

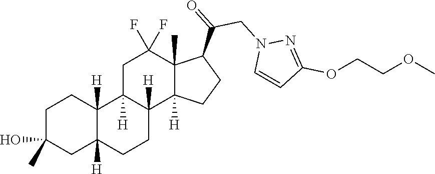

### Example 12

### tert-butyl 3-oxo-2,3-dihydro-1H-pyrazole-1-carboxylate

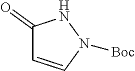 [SMILES: CC(C)(C)OC(=O)n1ccc(=O)[nH]1]

### Example 12-A

To a stirring suspension of 1H-pyrazol-3(2H)-one (1.0 g, 12 mmol) in DCM (10 mL) was added di-tert-butyl dicarbonate (2.8 g, 13 mmol) and TEA (1.3 g, 13 mmol) at r.t. Then the resulting mixture was allowed to stir at r.t. overnight. The reaction mixture was concentrated and purified by flash column chromatography (eluted with 1-5% MeOH in DCM) to Example 12-A (1.9 g, 86%) as a white solid. ¹H NMR (400 MHz, CDCl3) δ 12.39 (br. s, 1H), 7.82 (br. s, 1H), 5.90 (d, J=3.0 Hz, 1H), 1.63 (s, 9H).

### tert-butyl 3-(2-methoxyethoxy)-1H-pyrazole-1-carboxylate

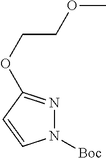 [SMILES: COCCOc1ccn(C(=O)OC(C)(C)C)n1]

### Example 12-B

To a stirred solution of Example 12-A (1.0 g, 5.5 mmol) and 1-bromo-2-methoxyethane (1.5 g, 11 mmol) in DMF (15 mL) was added NaI (0.82 g, 5.5 mmol) and K₂CO₃ (2.3 g, 16 mmol) at r.t. Then the resulting mixture was allowed to stir at 60° C. under N₂ overnight. The reaction mixture was cooled down to r.t, quenched with water (20 mL), extracted with EtOAc (3×20 mL). The combined organic phases were washed with brine, dried over Na₂SO₄, concentrated, and purified by flash column chromatography (silica gel, eluted with 5-12% EtOAc in heptane) to give Example 12-B (640 mg, 49%) as a yellow oil. ¹H NMR (400 MHz, CDCl3) δ 7.81 (d, 1H), 5.91 (d, 1H), 4.51-4.36 (m, 2H), 3.86-3.64 (m, 2H), 3.42 (s, 3H), 1.60 (s, 9H).

### 3-(2-methoxyethoxy)-1H-pyrazole

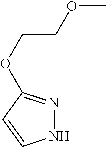

### Example 12-C

To a stirred solution of Example 12-B (0.64 g, 2.6 mmol) in DCM (5 mL) was added TFA (4 mL) at r.t. The reaction mixture was stirred at rt for 16 h and concentrated. The residue was dissolved in DCM (20 mL) and solid K₂CO₃ was added. The insoluble materials were removed by filtration and the filtrate was concentrated to give Example 12-C (240 mg, 64%) as a yellow oil. ¹H NMR (400 MHz, CDCl3) δ 7.35 (d, 1H), 5.76 (d, 1H), 4.42-4.26 (m, 2H), 3.80-3.69 (m, 2H), 3.43 (s, 3H).

### 1-((3R,5R,8R,9R,10S,13 S,14S,17S)-12,12-difluoro-3-hydroxy-3,13-dimethylhexadecahydro-1H-cyclopenta[a]phenanthren-17-yl)-2-(3-(2-methoxyethoxy)-1H-pyrazol-1-yl)ethan-1-one

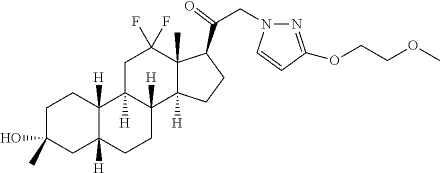

### Example 12

A mixture of Example 6-A (100 mg, 0.22 mmol), Example 12-C (28 mg, 0.2 mmol) and Cs₂CO₃ (143 mg, 0.44 mmol) in acetonitrile (5 mL) was allowed to stir at 30° C. under N₂ overnight. The reaction mixture was quenched with water (10 mL), and extracted with EtOAc (3×10 mL). The combined organic phases were washed with brine, dried over Na₂SO₄ and concentrated to give the crude product. The crude product was purified by flash column chromatography (silica gel, eluted with 10-50% EtOAc in heptane) to give Example 12 (45 mg, 45%) as a yellow viscous oil. ¹H NMR (400 MHz, CDCl3) δ 7.15 (d, 1H), 5.74 (d, 1H), 4.93 (d, 1H), 4.77 (d, 1H), 4.38-4.22 (m, 2H), 3.80-3.62 (m, 2H), 3.42 (s, 3H), 3.20 (t, 1H), 2.14-2.06 (m, 2H), 1.86-1.30 (m, 17H), 1.27 (s, 3H), 1.15-1.08 (m, 2H), 0.82 (s, 3H).

### 1-((3R,5R,8R,9R,10S,13 S,14S,17S)-12,12-difluoro-3-hydroxy-3,13-dimethylhexadecahydro-1H-cyclopenta[a]phenanthren-17-yl)-2-(3-(2-(2-methoxyethoxy)ethoxy)-1H-pyrazol-1-yl)ethan-1-one

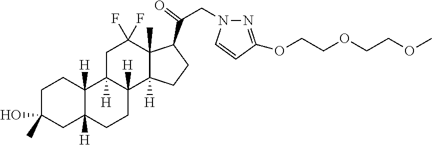

### Example 13

### tert-butyl 3-(2-(2-methoxyethoxy)ethoxy)-1H-pyrazole-1-carboxylate

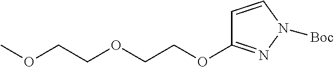 [SMILES: COCCOCCOc1ccn(C(=O)OC(C)(C)C)n1]

### Example 13-A

Example 13-A was made in the same fashion as compound Example 12-B substituting 1-bromo-2-methoxyethane with 1-bromo-2-(2-methoxyethoxy) ethane. ¹H NMR (400 MHz, CDCl3) δ 7.81 (d, 1H), 5.89 (d, 1H), 4.54-4.43 (m, 2H), 3.85-3.79 (m, 2H), 3.72-3.64 (m, 2H), 3.58-3.53 (m, 2H), 3.37 (s, 3H), 1.60 (s, 9H).

### 3-(2-(2-methoxyethoxy)ethoxy)-1H-pyrazole

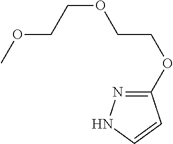

### Example 13-B

Example 13-B was made in the same fashion as Example 12-C substituting Example 12-B with Example 13-A. ¹H NMR (400 MHz, CDCl3) δ 7.35 (d, 1H), 5.75 (d, 1H), 4.44-4.26 (m, 2H), 3.87-3.83 (m, 2H), 3.75-3.67 (m, 2H), 3.63-3.57 (m, 2H), 3.39 (s, 3H).

### 1-((3R,5R,8R,9R,10S,13 S,14S,17S)-12,12-difluoro-3-hydroxy-3,13-dimethylhexadecahydro-1H-cyclopenta[a]phenanthren-17-yl)-2-(3-(2-(2-methoxyethoxy)ethoxy)-1H-pyrazol-1-yl)ethan-1-one

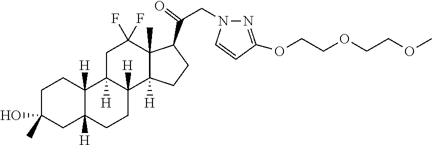

### Example 13

Example 13 was made in the same fashion as Example 12 substituting Example 12-C with Example 13-B. ¹H NMR (400 MHz, CDCl3) δ 7.15 (d, 1H), 5.72 (d, 1H), 4.92 (d, 1H), 4.76 (d, 1H), 4.28 (dd, 2H), 3.81 (dd, 2H), 3.69 (dd, 2H), 3.56 (dd, 2H), 3.38 (s, 3H), 3.20 (t, J=8.9 Hz, 1H), 2.17-2.05 (m, 2H), 1.86-1.30 (m, 17H), 1.27 (s, 3H), 1.15-1.08 (m, 2H), 0.82 (s, 3H).

### 1-((3R,5R,8R,9R,10S,13 S,14S,17S)-12,12-difluoro-3-hydroxy-3,13-dimethylhexadecahydro-1H-cyclopenta[a]phenanthren-17-yl)-2-(3-(2-(2-(2-methoxyethoxy)ethoxy)ethoxy)-1H-pyrazol-1-yl)ethan-1-one

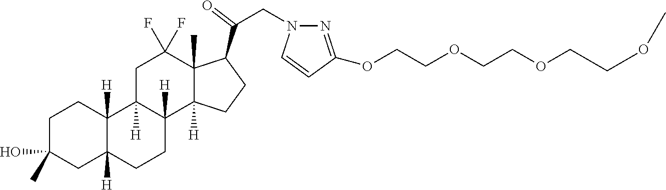

### Example 14

### Synthesis of 2-(2-(2-methoxyethoxy)ethoxy)ethyl Methanesulfonate

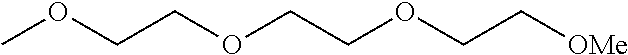

### Example 14-A

To a 250 mL round-bottom flask was added 2-(2-(2-methoxyethoxy)ethoxy)ethanol (5.0 g, 30 mmol), DIPEA (5.5 mL, 33 mmol) and DCM (80 mL). The mixture was cooled to 0° C. and methanesulfonyl chloride (2.6 mL, 33 mmol) was added via a syringe. The reaction mixture was allowed to stir for 1.5 h at 0° C. The reaction solution was transferred to a separatory funnel and washed with 100 mL of brine. The organic phase was concentrated under reduced pressure and the residue was partitioned between hexanes and water in a separatory funnel. The aqueous phase was separated, NaCl (10 g) was added, and the solution was extracted with DCM (3×50 mL). The combined organic phases were dried over Na₂SO₄, filtered, and concentrated to afford Example 14-A (7.4 g, quantitative yield) as a yellow oil. ¹H NMR (400 MHz, CDCl3) δ 4.42-4.28 (m, 2H), 3.79-3.71 (m, 2H), 3.71-3.58 (m, 6H), 3.57-3.47 (m, 2H), 3.36 (s, 3H), 3.07 (s, 3H).

### tert-butyl 3-(2-(2-(2-methoxyethoxy)ethoxy)ethoxy)-1H-pyrazole-1-carboxylate

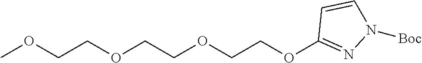 [SMILES: COCCOCCOCCOc1ccn(C(=O)OC(C)(C)C)n1]

### Example 14-B

Example 14-B was made in the same fashion as compound Example 12-B substituting 1-bromo-2-methoxyethane with Example 14-A. ¹H NMR (400 MHz, CDCl3) δ 7.75 (d, 1H), 5.83 (d, 1H), 4.45-4.32 (m, 2H), 3.84-3.73 (m, 2H), 3.65-3.56 (m, 6H), 3.52-3.45 (m, 2H), 3.31 (s, 3H), 1.54 (s, 9H).

### 3-(2-(2-(2-methoxyethoxy)ethoxy)ethoxy)-1H-pyrazole

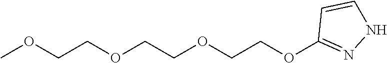 [SMILES: COCCOCCOCCOc1cc[nH]n1]

### Example 14-C

Example 14-C was made in the same fashion as Example 12-C substituting Example 12-B with Example 14-B. ¹H NMR (400 MHz, CDCl3) δ 7.36 (d, 1H), 5.75 (d, 1H), 4.42-4.29 (m, 2H), 3.85-3.82 (m, 2H), 3.75-3.63 (m, 6H), 3.57-3.50 (m, 2H), 3.37 (s, 3H).

### 1-((3R,5R,8R,9R,10S,13 S,14S,17S)-12,12-difluoro-3-hydroxy-3,13-dimethylhexadecahydro-1H-cyclopenta[a]phenanthren-17-yl)-2-(3-(2-(2-(2-methoxyethoxy)ethoxy)ethoxy)-1H-pyrazol-1-yl)ethan-1-one

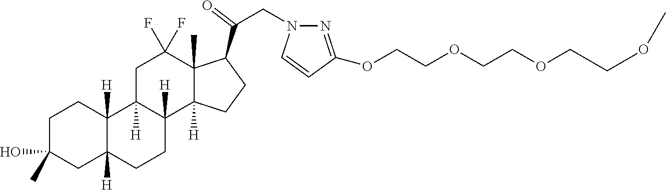

### Example 14

Example 14 was made in the same fashion as Example 12 substituting Example 12-C with Example 14-C. ¹H NMR (400 MHz, CDCl3) δ 7.15 (d, 1H), 5.72 (d, 1H), 4.93 (d, 1H), 4.77 (d, 1H), 4.27 (dd, 2H), 3.81 (dd, 2H), 3.74-3.63 (m, 6H), 3.55 (dd, 2H), 3.37 (s, 3H), 3.20 (t, 1H), 2.14-2.06 (m, 2H), 1.86-1.30 (m, 17H), 1.27 (s, 3H), 1.15-1.08 (m, 2H), 0.82 (s, 3H).

### 1-(2-((3R,5R,8R,9R,10S,13S,14S,17S)-12,12-difluoro-3-hydroxy-3,13-dimethylhexadecahydro-1H-cyclopenta[a]phenanthren-17-yl)-2-oxoethyl)-1H-1,2,3-triazole-4-carboxamide

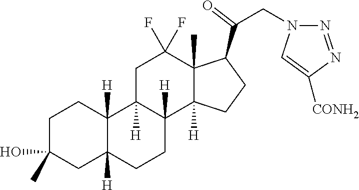

### Example 15

A mixture of Example 9-B (350 mg, 0.88 mmol), propiolamide (67 mg, 0.97 mmol), CuSO₄.5H2O (22 mg, 0.089 mmol) and L-Sodium ascorbate (87 mg, 0.44 mmol) in t-BuOH/H₂O (5 mL/5 mL) was allowed to stir at 40° C. under N₂ overnight. The reaction mixture was quenched by water (10 mL), extracted with DCM (3×20 mL). The combined organic phases were washed with brine, dried over Na₂SO₄ and concentrated to give the crude product. The crude product was purified by flash column chromatography (eluted with 2-10% MeOH in DCM) to give Example 15 (184 mg, 45%) as a white solid. ¹H NMR (400 MHz, DMSO-d₆) δ 8.33 (s, 1H), 7.89 (s, 1H), 7.50 (s, 1H), 5.60 (d, 1H), 5.51 (d, 1H), 4.35 (s, 1H), 3.30 (m, 1H), 2.02-1.97 (m, 2H), 1.92-1.13 (m, 19H), 1.11 (s, 3H), 0.80 (s, 3H).

### 1-(2-((3R,5R,8R,9R,10S,13S,14S,17S)-12,12-difluoro-3-hydroxy-3,13-dimethylhexadecahydro-1H-cyclopenta[a]phenanthren-17-yl)-2-oxoethyl)-1H-1,2,3-triazole-4-carbonitrile

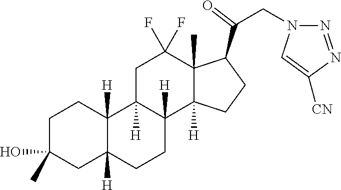

### Example 16

To a stirring suspension of Example 15 (100 mg, 0.22 mmol) in DCM (10 mL) was added pyridine (136 mg, 1.7 mmol) and trifluoroacetic acid anhydride (TFAA, 271 mg, 1.3 mmol) at r.t. under N₂. After the addition, the reaction mixture was allowed to stir for 1.5 h. The reaction mixture was diluted with DCM (20 mL), washed with brine, dried over Na₂SO₄, concentrated, and purified by flash column chromatography (silica gel, eluted with 5-20% EtOAc in heptane) to give Example 16 (100 mg, 85%) as a white solid. ¹H NMR (400 MHz, CDCl3) δ 8.03 (s, 1H), 5.63 (dd, 1H), 5.31 (d, 1H), 3.40 (t, 1H), 2.25-2.11 (m, 2H), 2.07-1.13 (m, 19H), 1.66 (s, 3H), 0.85 (s, 3H).

### 1-((3R,5R,8R,9R,10S,13 S,14S,17S)-3-hydroxy-3,13-dimethylhexadecahydro-1H-cyclopenta[a]phenanthren-17-yl)-2-(4-((2-methoxyethoxy)methyl)-1H-1,2,3-triazol-1-yl)ethan-1-one

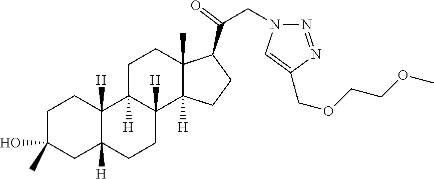

### Example 17

### 2-azido-1-((3R,5R,8R,9R,10S,13S,14S,17S)-3-hydroxy-3,13-dimethylhexadecahydro-1H-cyclopenta[a]phenanthren-17-yl)ethanone

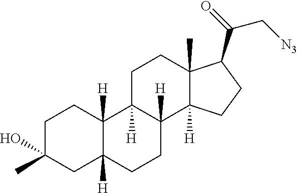

### Example 17-A

A mixture of known compound 2-bromo-1-((3R,5R,8R,9R,10S,13S,14S,17S)-3-hydroxy-3,13-dimethylhexadecahydro-1H-cyclopenta[a]phenanthren-17-yl)ethanone (470 mg, 1.18 mmol) and sodium azide (154 mg, 2.36 mmol) in MeOH (10 mL) was allowed to stir at r.t. under N₂ for 1 h. The reaction mixture was poured into water (20 mL), extracted with EtOAc (3×20 mL). The combined organic phases were washed with brine, dried over Na₂SO₄, and concentrated to give the crude Example 17-A (450 mg, 100%) as white solid. ¹H NMR (400 MHz, CDCl3) δ 3.88 (s, 2H), 2.50 (t, J=9.0 Hz, 1H), 2.30-2.10 (m, 1H), 1.91-1.02 (m, 22H), 1.27 (s, 3H), 0.65 (s, 3H).

### 1-((3R,5R,8R,9R,10S,13 S,14S,17S)-3-hydroxy-3,13-dimethylhexadecahydro-1H-cyclopenta[a]phenanthren-17-yl)-2-(4-((2-methoxyethoxy)methyl)-1H-1,2,3-triazol-1-yl)ethan-1-one

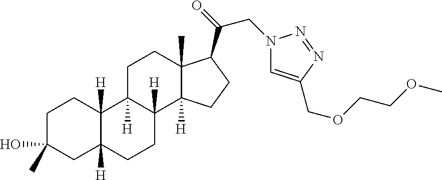

### Example 17

A mixture of Example 17-A (100 mg, 0.28 mmol), Example 9-A (38 mg, 0.33 mmol), CuSO₄.5H₂O (7 mg, 0.03 mmol) and L-Sodium ascorbate (22 mg, 0.11 mmol) in THF/H₂O (8 mL/2 mL) was allowed to stir at 40° C. under N₂ for 2 h. The reaction mixture was quenched with water (10 mL) and extracted with EtOAc (3×10 mL). The combined organic phases were washed with brine, dried over Na₂SO₄, concentrated, and purified by flash column chromatography (silica gel, eluted with 1-5% MeOH in DCM) to give Example 17 (50 mg, 38%) as a yellow viscous oil. ¹H NMR (400 MHz, CDCl3) δ 7.64 (s, 1H), 5.21 (d, 1H), 5.09 (d, 1H), 4.72 (s, 2H), 3.74-3.65 (m, 2H), 3.59-3.52 (m, 2H), 3.38 (s, 3H), 2.64 (t, J=8.8 Hz, 1H), 2.26-2.15 (m, 1H), 2.07 (dt, J=12.0, 2.8 Hz, 1H), 1.88-1.32 (m, 18H), 1.27 (s, 3H), 1.15-1.05 (m, 3H), 0.67 (s, 3H).

### 1-((3R,5R,8R,9R,10S,13 S,14S,17S)-3-hydroxy-3,13-dimethylhexadecahydro-1H-cyclopenta[a]phenanthren-17-yl)-2-(4-((2-(2-methoxyethoxy)ethoxy)methyl)-1H-1,2,3-triazol-1-yl)ethan-1-one

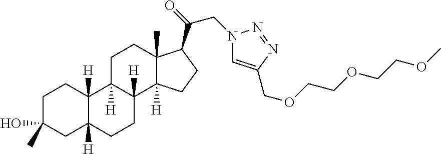

### Example 18

Example 18 was made in the same fashion as Example 17 substituting Example 9-A with Example 10-A. ¹H NMR (400 MHz, CDCl3) δ 7.65 (s, 1H), 5.21 (d, 1H), 5.09 (d, 1H), 4.72 (s, 2H), 3.77-3.61 (m, 6H), 3.55 (dd, 2H), 3.37 (s, 3H), 2.64 (t, 1H), 2.21 (q, 1H), 2.07 (d, 1H), 1.88-1.32 (m, 18H), 1.27 (s, 3H), 1.14-1.06 (m, 3H), 0.67 (s, 3H).

### 2-(4-(2,5,8,11-tetraoxadodecyl)-1H-1,2,3-triazol-1-yl)-1-((3R,5R,8R,9R,10S,13S,14S,17S)-3-hydroxy-3,13-dimethylhexadecahydro-1H-cyclopenta[a]phenanthren-17-yl)ethan-1-one

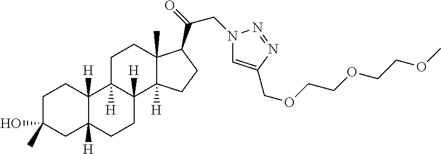

### Example 19

Example 19 was made in the same fashion as Example 17 substituting Example 9-A with Example 11-A. ¹H NMR (400 MHz, CDCl3) δ 7.67 (s, 1H), 5.22 (d, 1H), 5.11 (d, 1H), 4.81 (d, 1H), 4.72 (s, 2H), 3.73-3.60 (m, 10H), 3.58-3.49 (m, 2H), 3.37 (s, 3H), 2.64 (t, 1H), 2.20 (q, 1H), 2.07 (dt, 1H), 1.87-1.32 (m, 18H), 1.27 (s, 3H), 1.15-1.06 (m, 3H), 0.67 (s, 3H).

### 1-((3R,5R,8R,9R,10S,13S,14S,17S)-3-hydroxy-3,13-dimethylhexadecahydro-1H-cyclopenta[a]phenanthren-17-yl)-2-(3-(2-methoxyethoxy)-1H-pyrazol-1-yl)ethan-1-one

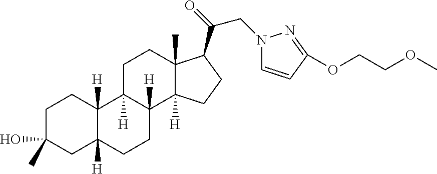

### Example 20

A mixture of 2-bromo-1-((3R,5R,8R,9R,10S,13S,14S,17S)-3-hydroxy-3,13-dimethylhexadecahydro-1H-cyclopenta[a]phenanthren-17-yl)ethanone (200 mg, 0.50 mmol), Example 12-C (71 mg, 0.50 mmol), and Cs₂CO₃ (328 mg, 1.0 mmol) in acetonitrile (5 mL) was allowed to stir at r.t. under N₂ overnight. The reaction mixture was quenched with water (10 mL) and extracted with EtOAc (3×10 mL). The combined organic phases were washed with brine, dried over Na₂SO₄, concentrated, and the residue was purified by flash column chromatography (silica gel, eluted with 10-50% EtOAc in heptane) to give Example 20 (132 mg, 57%) as a yellow viscous oil. ¹H NMR (400 MHz, CDCl3) δ 7.18 (d, 1H), 5.75 (d, 1H), 4.72 (d, 1H), 4.63 (d, 1H), 4.32-4.22 (m, 2H), 3.84-3.65 (m, 2H), 3.42 (s, 3H), 2.54 (t, 1H), 2.23-2.11 (m, 1H), 2.02 (dt, 1H), 1.87-1.22 (m, 18H), 1.27 (s, 3H), 1.13-1.04 (m, 3H), 0.66 (s, 3H).

### 1-((3R,5R,8R,9R,10S,13S,14S,17S)-3-hydroxy-3,13-dimethylhexadecahydro-1H-cyclopenta[a]phenanthren-17-yl)-2-(3-(2-(2-methoxyethoxy)ethoxy)-1H-pyrazol-1-yl)ethan-1-one

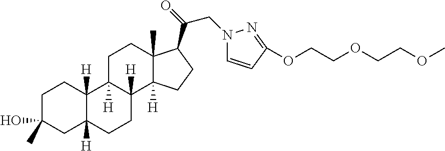

### Example 21

Example 21 was made in the same fashion as Example 20 substituting Example 12-C with Example 13-B. ¹H NMR (400 MHz, CDCl3) δ 7.17 (d, 1H), 5.73 (d, 1H), 4.71 (d, 1H), 4.62 (d, 1H), 4.31-4.23 (m, 2H), 3.87-3.79 (m, 2H), 3.71-3.65 (m, 2H), 3.60-3.53 (m, 2H), 3.38 (s, 3H), 2.54 (t, 1H), 2.22-2.12 (m, 1H), 2.01 (dt, 1H), 1.87-1.22 (m, 18H), 1.26 (s, 3H), 1.13-1.04 (m, 3H), 0.66 (s, 3H).

### 1-((3R,5R,8R,9R,10S,13S,14S,17S)-3-hydroxy-3,13-dimethylhexadecahydro-1H-cyclopenta[a]phenanthren-17-yl)-2-(3-(2-(2-(2-methoxyethoxy)ethoxy)ethoxy)-1H-pyrazol-1-yl)ethan-1-one

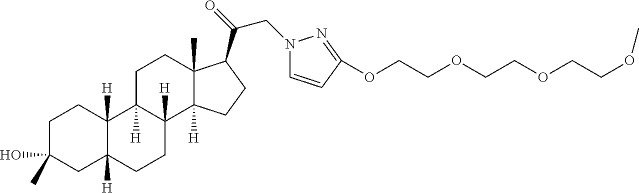

### Example 22

Example 22 was made in the same fashion as Example 20 substituting Example 12-C with Example 14-C. ¹H NMR (400 MHz, CDCl3) δ 7.18 (d, 1H), 5.74 (d, 1H), 4.72 (d, 1H), 4.64 (d, 1H), 4.33-4.21 (m, 2H), 3.86-3.79 (m, 2H), 3.74-3.61 (m, 6H), 3.59-3.51 (m, 2H), 3.38 (s, 3H), 2.55 (t, 1H), 2.17 (m, 1H), 2.10-1.97 (m, 1H), 1.87-1.22 (m, 18H), 1.27 (s, 3H), 1.13-1.04 (m, 3H), 0.66 (s, 3H).

### 3-(2-((3R,5R,8R,9R,10S,13S,14S,17S)-3-hydroxy-3,13-dimethylhexadecahydro-1H-cyclopenta[a]phenanthren-17-yl)-2-oxoethyl)benzo[d]oxazol-2(3H)-one

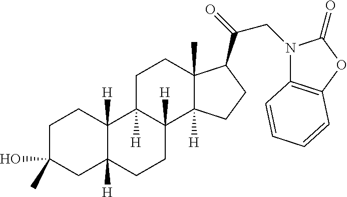

### Example 23

Example 23 was made in the same fashion as Example 20 substituting Example 12-C with benzo[d]oxazol-2(3H)-one. ¹H NMR (400 MHz, CDCl3) δ 7.23 (d, 1H), 7.10-7.17 (m, 2H), 6.75 (d, 1H), 4.49 (d, 1H), 4.63 (d, 1H), 2.66 (t, 1H), 2.26-2.10 (m, 2H), 1.10-1.88 (m, 21H), 1.28 (s, 3H), 0.71 (s, 3H).

### 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-3-methyl-1H-benzo[d]imidazol-2(3H)-one

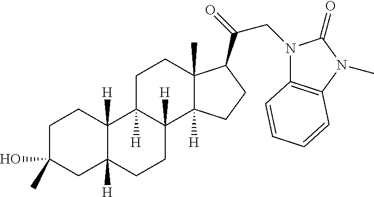

### Example 24

Example 24 was made in the same fashion as Example 20 substituting Example 12-C with 1-methyl-1H-benzo[d]imidazol-2(3H)-one. ¹H NMR (400 MHz, CDCl3) δ7.02-7.11 (m, 2H), 6.98 (d, 1H), 6.74 (d, 1H), 4.55 (d, 1H), 4.67 (d, 1H), 3.42 (s, 3H), 2.66 (t, 1H), 2.16-2.24 (m, 2H), 1.08-1.87 (m, 21H), 1.27 (s, 3H), 0.70 (s, 3H).

### 2-(2-((3R,5R,8R,9R,10S,13S,14S,17S)-3-hydroxy-3,13-dimethylhexadecahydro-1H-cyclopenta[a]phenanthren-17-yl)-2-oxoethyl)benzo[d]isoxazol-3(2H)-one

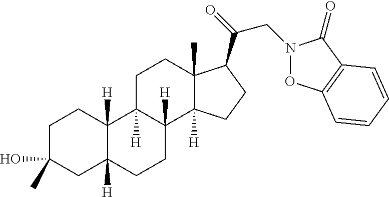

### Example 25

Example 25 was made in the same fashion as Example 20 substituting Example 12-C with benzo[d]isoxazol-3(2H)-one. ¹HNMR (400 MHz, CDCl3) δ7.72 (d, 1H), 7.54 (t, 1H), 7.42 (d, 1H), 7.29 (t, 1H), 4.91 (d, 1H), 5.08 (d, 1H), 2.67 (t, 1H), 2.18-2.21 (m, 1H), 2.00-2.13 (m, 1H), 1.61-11.86 (m, 10H), 1.05-1.48 (m, 11H), 1.26 (s, 3H), 0.71 (s, 3H).

### 2-(2-((3R,5R,8R,9R,10S,13S,14S,17S)-3-hydroxy-3,13-dimethylhexadecahydro-1H-cyclopenta[a]phenanthren-17-yl)-2-oxoethyl)isoxazol-3(2H)-one

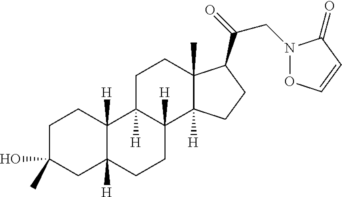

### Example 26

Example 26 was made in the same fashion as Example 20 substituting Example 12-C with isoxazol-3(2H)-one. ¹H NMR (400 MHz, CDCl3) δ8.14 (s, 1H), 6.08 (s, 1H), 4.77 (d, 1H), 4.91 (d, 1H), 2.61 (t, J=8.7 Hz, 1H), 2.19-2.27 (m, 1H), 2.00-2.13 (m, 1H), 1.64-1.86 (m, 9H), 1.07-1.49 (m, 12H), 1.26 (s, 3H), 0.68 (s, 3H).

### Example 27. GABA_{A}R Subunit Expression and GABA_{A}R Modulator FLIPR Assay

293T cells (ATCC-CRL-1573) were applied for transient expression of α1β2γ2 or α1β2δ GABA_{A} receptors. Cells were plated in a 96 well plate with a confluence of about 70%-80% and cultured overnight in complete medium without antibiotics. The α1β2γ2 or α1β2δ subunits were transfected via the lipofectamine method. The FLIPR assay was developed to quantitatively assess the compounds' PAM activity in α1β2γ2 and α1β2δ GABA_{A} receptors. The FLIPR assays were conducted 48 h post-transfection. The FLIPR reagents were purchased from Molecular Devices Corporation (Membrane potential red kit, Catalog number R8126). The FMP-Red-Dye was reconstituted to 1× assay buffer. Growth medium was removed from the wells and cells were loaded with 100 μL FMP-Red-Due solution for 30 minutes in the dark at r.t. After dye loading, each plate was transferred to the FLIPR Tetra Station: the dye was excited at 510-545 nm, and the fluorescence signals were recorded at 565-625 nm. Baseline recordings were acquired for 1 minute. FMP-Red-Dye is a slow-response, potential sensitive probe and the maximal response is usually seen within 2 minutes after triggering cellular depolarization or hyperpolarization. After baseline recording, the different doses of compound solutions were firstly added on cells and the fluorescent signals were recorded for 5 minutes. Then the cells were followed by a second addition with ligand GABA solution and recorded for another 3.5 minutes. Cell responses were calculated as ΔF=Fₘₐₓ−Fₘᵢₙ, where Fₘₐₓ was the maximum response in arbitrary fluorescence units, and Fₘᵢₙ was the baseline arbitrary fluorescence unit value.

### Example 28. The Activity of Selected Compounds in the FLIPR Assay

The EC₅₀ values for selected compounds in α1β2γ2 and α1β2δ are shown in Table 1.

TABLE 1
EC₅₀ values for selected compounds in 293T cells transiently
expressed α1β2γ2 and α1β2δ GABA_{A} receptors

| Examples | EC₅₀ in α1β2γ2/293T (μM) | EC₅₀ in α1β2δ/293T (μM) |
|---|---|---|
| 1 | 0.27 | 0.081 |
| 2 | 0.28 | 0.098 |
| 4 | 2.95 | 0.71 |
| 5 | 0.39 | 0.19 |
| 6 | 0.47 | 0.12 |
| 7 | 0.74 | 0.39 |
| 8 | 0.63 | 0.25 |

### Example 29. The Method of Determining Brain and Plasma Exposure of Example 8 in Mice

CD1 mice were dosed at 10 mg/kg via oral gavage with Example 8. Approximately 2 milliliters of blood sample were withdrawn with a 21 G needle via cardiac puncture into K2-EDTA treated tubes sampling at 20 min, 40 min, 1 h, 2 h, 4 h, and 8 h. The blood samples were centrifuged at 2000 g for 15 min and frozen at −80° C. until extraction. The whole brain samples were harvested sampling at 20 min, 40 min, 1 h, 2 h, 4 h, and 8 h, washed with saline, and frozen at −80° C. until homogenization/extraction. A validated method using liquid chromatography/tandem weight spectrometry (LC/MS/MS) was used to analyze all samples.

### Example 30. The Brain and Plasma Exposure of Example 8 in Mice

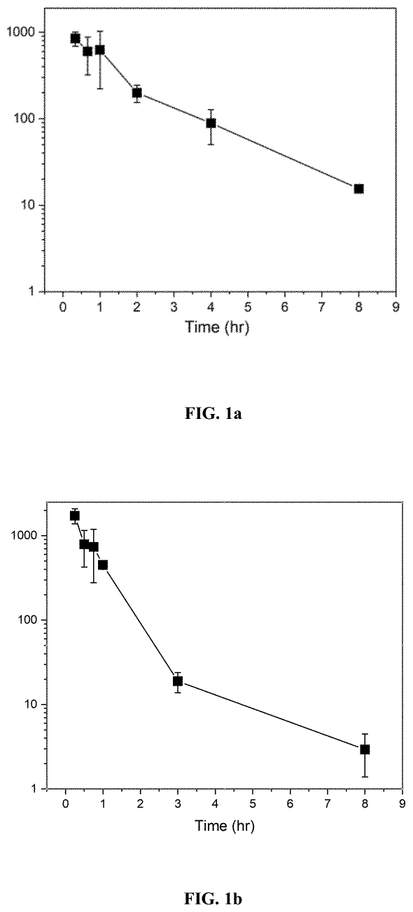

The brain (FIG. 1a) and plasma (FIG. 1b) exposure (ng/mL) of Example 8 in mice (10 mg/kg, p.o.) were conducted with results shown in FIG. 1.

### Example 31. The Protocols for the Open Field Test of Example 8 in Mice

9-month old 5XFAD transgenic mice (expressing human APP and PSEN1 transgenes with 5 AD-linked mutations) and wild type litermates (C57/B16) were dosed as at 20 mg/kg via oral gavage. The total distance travelled was recorded in an open field chamber 45 minutes post dosing.

### Example 32. The Open Field Test of Example 8 in 5XFAD and Wild Type (WT) Mice

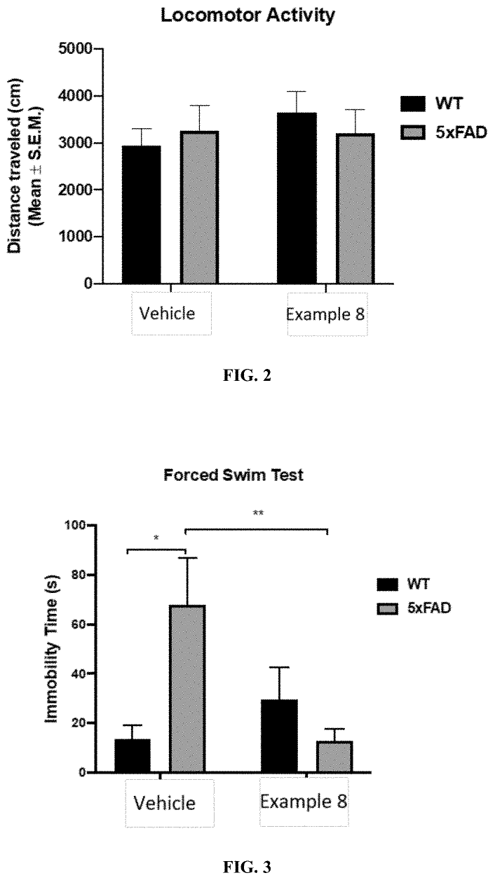

Example 8 showed no interference of the locomotor functions in this test (FIG. 2).

### Example 33. The Protocols for the Behavioral Tests of Example 8 in Mice

9-month old 5XFAD transgenic mice (expressing human APP and PSEN1 transgenes with 5 AD-linked mutations) and wild type litermates (C57/B16) were dosed as at 20 mg/kg via oral gavage. The cognitive and mood behavioral tests were performed 45 minutes post dosing.

### Example 34. The Forced Swim Test of Example 8 in 5XFAD and Wild Type (WT) Mice

The forced swim test is a test for depression-like behavior in rodents. The assay involves observing a mouse stay afloat in a tank of water and monitoring how much time it spends immobile. Example 8 demonstrated a genotype-specific efficacy in this model (FIG. 3).

### Example 35. The Tail Suspension Test of Example 8 in 5XFAD and Wild Type (WT) Mice

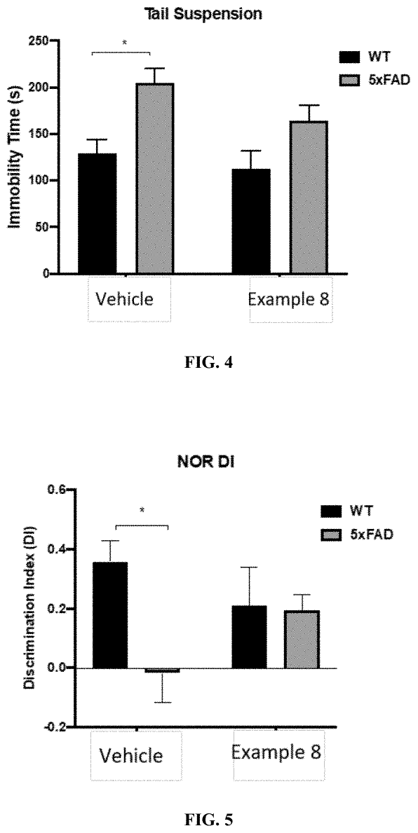

The tail suspension test is based on the fact that short-term, inescapable stress of suspension of animals by their tails leads to an immobile posture. Immobility time was measured for each group (FIG. 4).

### Example 36. The Novel Object Recognition (NOR) Test of Example 8 in 5XFAD and Wild Type (WT) Mice

The Novel Object Recognition (NOR) task was used to evaluate cognition, particularly recognition memory, in rodent models (FIG. 5). This test is based on the spontaneous tendency of rodents to spend more time exploring a novel object than a familiar one. The choice to explore the novel object (measured as discrimination index) reflects the use of learning and recognition memory.

### Example 37. The Social Recognition Test of Example 8 in 5XFAD and Wild Type (WT) Mice

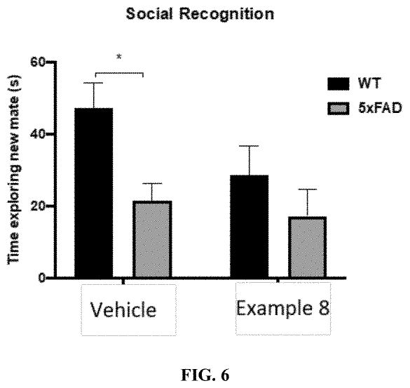

The social recognition test was performed, which was based on the natural tendency of mice to explore a novel congener instead of a familiar one. In this task, a first phase (Day 1) consisted of habituating the mouse to the experimental arena. In the second phase, the experimental mouse was presented to a congener. In the third phase (Day 3), the experimental subject was exposed to two congeners, including the familiar one and a novel one. Observations of the time the experimental mouse explored the novel congener was recorded (FIG. 6).

Another embodiment of the present invention is (3R,5R,8R,9R,10S,13S,14S,17S)-3-hydroxy-3,13-dimethyl-12-methylenehexadecahydro-1H-cyclopenta[a]phenanthrene-17-carbonitrile.

Another embodiment of the present invention is 1-((3R,5R,8R,9R,10S,13S,14S,17S)-3-hydroxy-3,13-dimethyl-12-methylenehexadecahydro-1H-cyclopenta[a]phenanthren-17-yl)ethan-1-one.

Another embodiment of the present invention is (3R,5R,8R,9R,10S,13S,14S,17S)-12,12-difluoro-3-hydroxy-3,13-dimethylhexadecahydro-1H-cyclopenta[a]phenanthrene-17-carboxylic acid.

Another embodiment of the present invention is (3R,5R,8R,9R,10S,13S,14S,17S)-12,12-difluoro-3-hydroxy-3,13-dimethylhexadecahydro-1H-cyclopenta[a]phenanthrene-17-carbonitrile.

Another embodiment of the present invention is 1-((3R,5R,8R,9R,10S,13S,14S,17S)-12,12-difluoro-3-hydroxy-3,13-dimethylhexadecahydro-1H-cyclopenta[a]phenanthren-17-yl)ethan-1-one.

Another embodiment of the present invention is 1-(2-((3R,5R,8R,9R,10S,13S,14S,17S)-12,12-difluoro-3-hydroxy-3,13-dimethylhexadecahydro-1H-cyclopenta[a]phenanthren-17-yl)-2-oxoethyl)-1H-pyrazole-4-carbonitrile.

Another embodiment of the present invention is 1-((3R,5R,8R,9R,10S,13S,14S,17S)-12,12-difluoro-3-hydroxy-3,13-dimethylhexadecahydro-1H-cyclopenta[a]phenanthren-17-yl)-2-(4-fluoro-1H-pyrazol-1-yl)ethan-1-one.

Another embodiment of the present invention is 1-(2-((3R,5R,8R,9R,10S,13S,14S,17S)-12,12-difluoro-3-hydroxy-3,13-dimethylhexadecahydro-1H-cyclopenta[a]phenanthren-17-yl)-2-oxoethyl)-1H-pyrazole-3-carbonitrile.

Another embodiment of the present invention is 1-((3R,5R,8R,9R,10S,13S,14S,17S)-12,12-difluoro-3-hydroxy-3,13-dimethylhexadecahydro-1H-cyclopenta[a]phenanthren-17-yl)-2-(4-((2-methoxyethoxy)methyl)-1H-1,2,3-triazol-1-yl)ethan-1-one.

Another embodiment of the present invention is 1-((3R,5R,8R,9R,10S,13S,14S,17S)-12,12-difluoro-3-hydroxy-3,13-dimethylhexadecahydro-1H-cyclopenta[a]phenanthren-17-yl)-2-(4-42-(2-methoxyethoxy)ethoxy)methyl)-1H-1,2,3-triazol-1-yl)ethan-1-one.

Another embodiment of the present invention is 2-(4-(2,5,8,11-tetraoxadodecyl)-1H1,2,3-triazol-1-yl)-1-((3R,5R,8R,9R,10S,13 S,14S,17S)-12,12-difluoro-3-hydroxy-3,13-dimethylhexadecahydro-1H-cyclopenta[a]phenanthren-17-yl)ethan-1-one.

Another embodiment of the present invention is 1-((3R,5R,8R,9R,10S,13S,14S,17S)-12,12-difluoro-3-hydroxy-3,13-dimethylhexadecahydro-1H-cyclopenta[a]phenanthren-17-yl)-2-(3 (2-methoxyethoxy)-1H-pyrazol-1-yl)ethan-1-one.

Another embodiment of the present invention is 1-((3R,5R,8R,9R,10S,13S,14S,17S)-12,12-difluoro-3-hydroxy-3,13-dimethylhexadecahydro-1H-cyclopenta[a]phenanthren-17-yl)-2-(3 (2-(2-methoxyethoxy)ethoxy)-1H-pyrazol-1-yl)ethan-1-one.

Another embodiment of the present invention is 1-((3R,5R,8R,9R,10S,13S,14S,17S)-12,12-difluoro-3-hydroxy-3,13-dimethylhexadecahydro-1H-cyclopenta[a]phenanthren-17-yl)-2-(3 (2-(2-(2-methoxyethoxy)ethoxy)ethoxy)-1H-pyrazol-1-yl)ethan-1-one.

Another embodiment of the present invention is 1-(2-((3R,5R,8R,9R,10S,13S,14S,17S)-12,12-difluoro-3-hydroxy-3,13-dimethylhexadecahydro-1H-cyclopenta[a]phenanthren-17-yl)-2-oxo ethyl)-1H-1,2,3-triazole-4-carboxamide.

Another embodiment of the present invention is 1-(2-((3R,5R,8R,9R,10S,13S,14S,17S)-12,12-difluoro-3-hydroxy-3,13-dimethylhexadecahydro-1H-cyclopenta[a]phenanthren-17-yl)-2-oxoethyl)-1H-1,2,3-triazole-4-carbonitrile.

Another embodiment of the present invention is 1-((3R,5R,8R,9R,10S,13S,14S,17S)-3-hydroxy-3,13-dimethylhexadecahydro-1H-cyclopenta[a]phenanthren-17-yl)-2-(4-((2-methoxyethoxy)methyl)-1H-1,2,3-triazol-1-yl)ethan-1-one.

Another embodiment of the present invention is 1-((3R,5R,8R,9R,10S,13S,14S,17S)-3-hydroxy-3,13-dimethylhexadecahydro-1H-cyclopenta[a]phenanthren-17-yl)-2-(4-((2-(2-methoxyethoxy)ethoxy)methyl)-1H-1,2,3-triazol-1-yl)ethan-1-one.

Another embodiment of the present invention is 2-(4-(2,5,8,11-tetraoxadodecyl)-1H1,2,3-triazol-1-yl)-1-((3R,5R,8R,9R,10S,13 S,14S,17S)-3-hydroxy-3,13-dimethylhexadecahydro-1H-cyclopenta[a]phenanthren-17-yl)ethan-1-one.

Another embodiment of the present invention is 1-((3R,5R,8R,9R,10S,13S,14S,17S)-3-hydroxy-3,13-dimethylhexadecahydro-1H-cyclopenta[a]phenanthren-17-yl)-2-(3-(2-methoxyethoxy)-1H-pyrazol-1-yl)ethan-1-one.

Another embodiment of the present invention is 1-((3R,5R,8R,9R,10S,13S,14S,17S)-3-hydroxy-3,13-dimethylhexadecahydro-1H-cyclopenta[a]phenanthren-17-yl)-2-(3-(2-(2-methoxyethoxy)ethoxy)-1H-pyrazol-1-yl)ethan-1-one.

Another embodiment of the present invention is 1-((3R,5R,8R,9R,10S,13S,14S,17S)-3-hydroxy-3,13-dimethylhexadecahydro-1H-cyclopenta[a]phenanthren-17-yl)-2-(3-(2-(2-(2-methoxyethoxy)ethoxy)ethoxy)-1H-pyrazol-1-yl)ethan-1-one.

Another embodiment of the present invention is 3-(2-((3R,5R,8R,9R,10S,13S,14S,17S)-3-hydroxy-3,13-dimethylhexadecahydro-1H-cyclopenta[a]phenanthren-17-yl)-2-oxoethyl)benzo[d]oxazol-2(3H)-one.

Another embodiment of the present invention is 1-(2-((3R,5R,8R,9R,10S,13S,14S,17S)-3-hydroxy-3,13-dimethylhexadecahydro-1H-cyclopenta[a]phenanthren-17-yl)-2-oxo ethyl)-3-methyl-1H-benzo [d]imidazol-2(3H)-one.

Another embodiment of the present invention is 2-(2-((3R,5R,8R,9R,10S,13S,14S,17S)-3-hydroxy-3,13-dimethylhexadecahydro-1H-cyclopenta[a]phenanthren-17-yl)-2-oxo ethyl)benzo[d]isoxazol-3 (2H)-one.

2-(2-((3R,5R,8R,9R,10S,13 S,14S,17S)-3-hydroxy-3,13-dimethylhexadecahydro-1H-cyclopenta[a]phenanthren-17-yl)-2-oxoethyl)isoxazol-3 (2H)-one.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A compound having the structural formula (I): 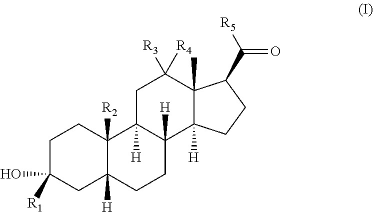
wherein
R₁ is H or a substituted or unsubstituted C₁-C₆ alkyl;
R₂ is H or a substituted or unsubstituted C₁-C₆ alkyl;
each of R₃ and R₄ is independently selected from the group consisting of H, halogen, a substituted or unsubstituted C₁-C₆ alkyl, optionally R₃ and R₄, along with the carbon to which they are attached, may form
an exocyclic double bond, or
a C₃-C₁₈-membered ring optionally substituted with one or more substituents selected from the group consisting of halogen, OH, CN, C₁-C₅ alkyl, and O—C₁-C₅ alkyl;
R₅ is OR′ or a C₁-C₆ alkyl optionally substituted with heterocyclic or heterobicyclic group, which is optionally substituted with one or more of CN, OH, halogen, a substituted or unsubstituted C₁-C₆ alkyl; provided that, if each of R₃ and R₄ is H, R₅ is a C₁ alkyl substituted with a heterocyclic or heterobicyclic group, which is optionally substituted with C═O(NR^{f}R^{g}), C(R^{f})(R^{g})(OR^{h}), or OR^{h}, wherein each of R′, R^{f} and R^{g} is independently selected from the group consisting of H, a substituted or unsubstituted C₁-C₆ alkyl, and R^{h} is (CH₂CH₂O)ₙCH₃ or CH₂O(CH₂CH₂O)ₙCH₃,
wherein n is 1, 2, 3, 4 or 5,
or a pharmaceutically acceptable form or an isotope derivative thereof.

2. The compound of claim 1, wherein at least one of R₃ and R₄ is a halogen.

3. (canceled)

4. The compound of claim 2, wherein the halogen is F.

5. The compound of claim 1, wherein each of R₃ and R₄ is H

6. The compound of claim 1, wherein R₁ is CH₃.

7. The compound of claim 1, wherein R₂ is H.

8. The compound of claim 1, wherein R₅ is an unsubstituted C₁-C₃ alkyl.

9. The compound of claim 8, wherein R₅ is an unsubstituted methyl.

10. The compound of claim 8, wherein R₅ is a substituted C₁-C₃ alkyl.

11. The compound of claim 10, wherein R₅ is a substituted methyl which is substituted with a heterocyclic or heterobicyclic group.

12. (canceled)

13. The compound of claim 1, wherein R₅ is CH₂R^{j}, wherein is selected from the group consisting of: 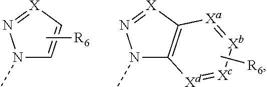
wherein
each of X, X^{a}, X^{b}, X^{c} and X^{d} is independently selected from N and CH; and
R₆ is CN, halogen, C═O(NR^{f}R^{g}), C(R^{f})(R^{g})(OR^{h}), or OR^{h}, wherein each of R^{f} and R^{g} is independently selected from the group consisting of H, a substituted or unsubstituted C₁-C₆ alkyl, and R^{h} is (CH₂CH₂O)ₙCH₃ or CH₂O(CH₂CH₂O)ₙCH₃,
wherein n is 1, 2, 3, 4 or 5.

14. The compound of claim 13, wherein X is CH.

15. The compound of claim 13, wherein X is N.

16. The compound of claim 13, wherein each of X^{a}, X^{b}, X^{c} and X^{d} is CH.

17. The compound of claim 13, wherein R₆ is CN, F, is C═O(NH₂, O(CH₂CH₂O)ₙCH₃ or CH₂O(CH₂CH₂O)ₙCH₃.

18-21. (canceled)

22. A compound selected from:

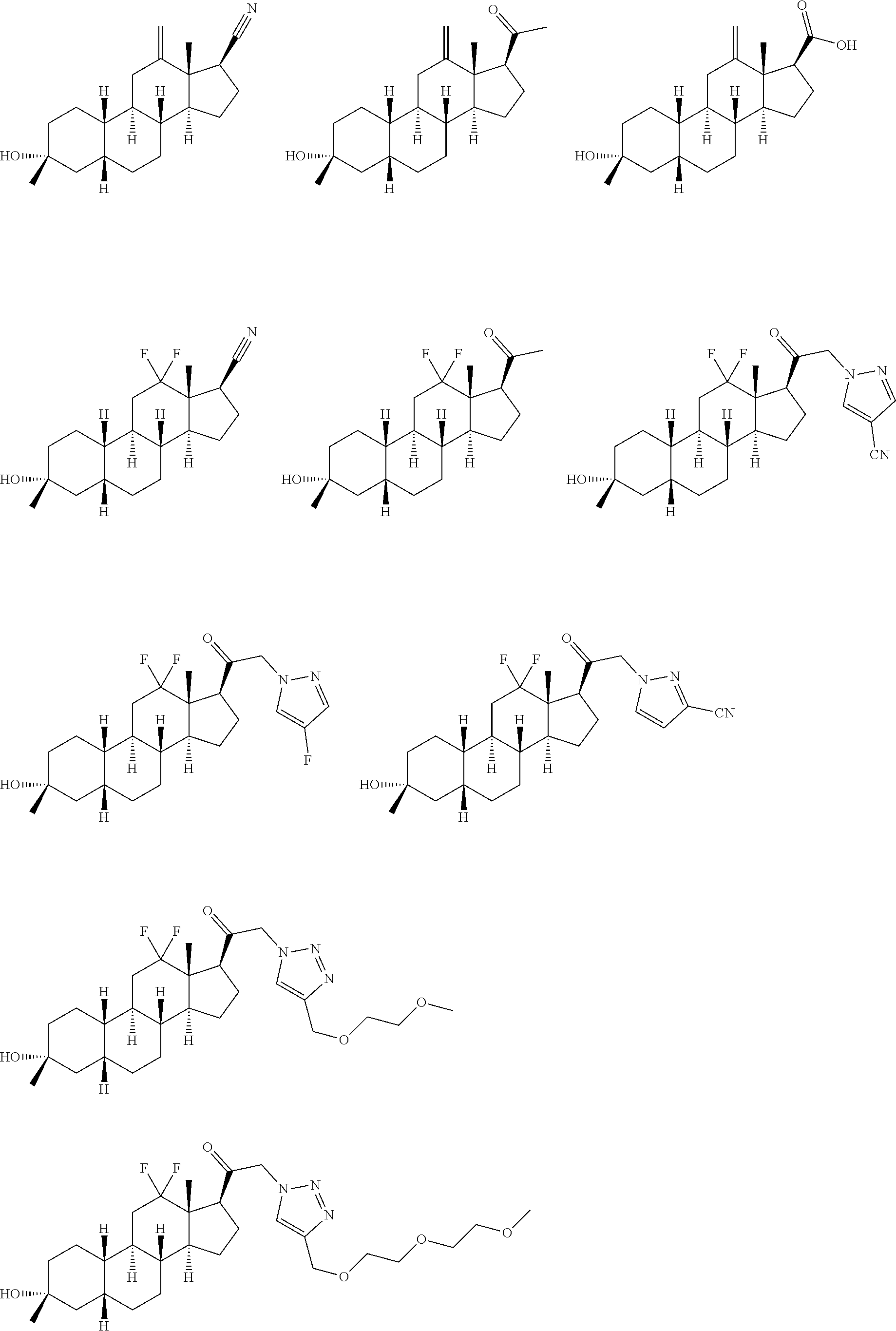

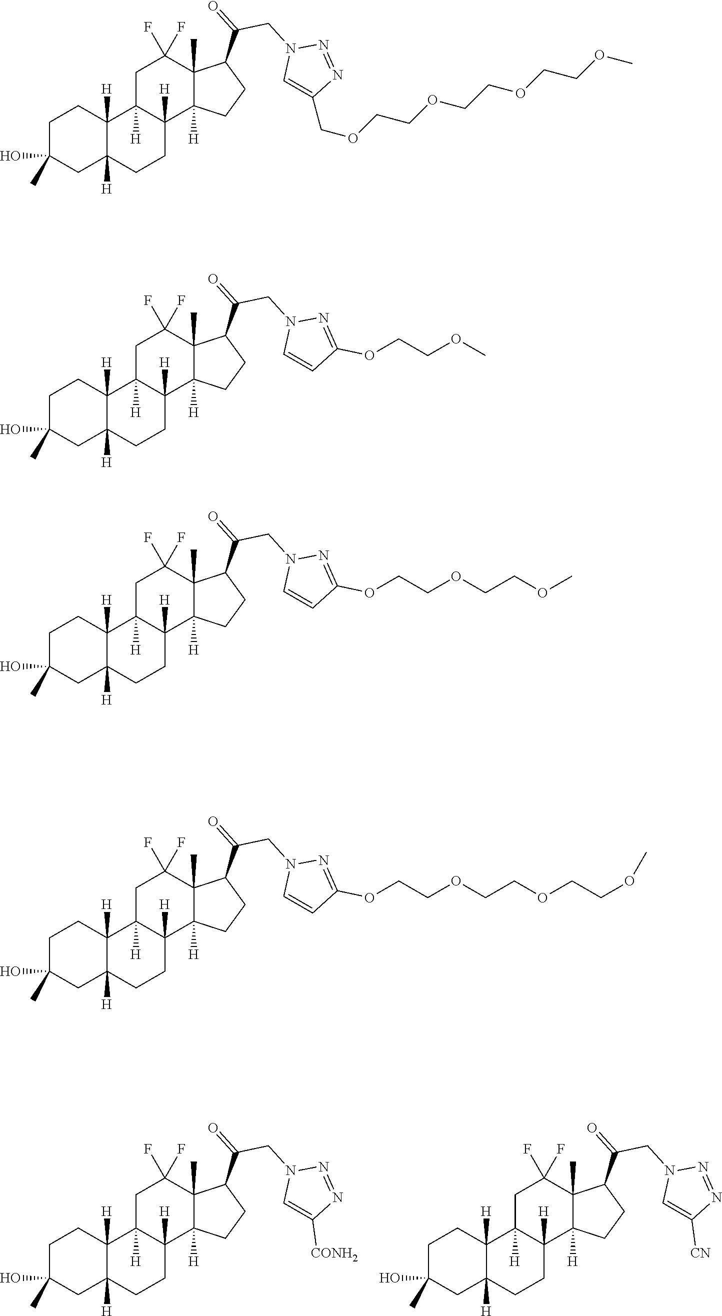

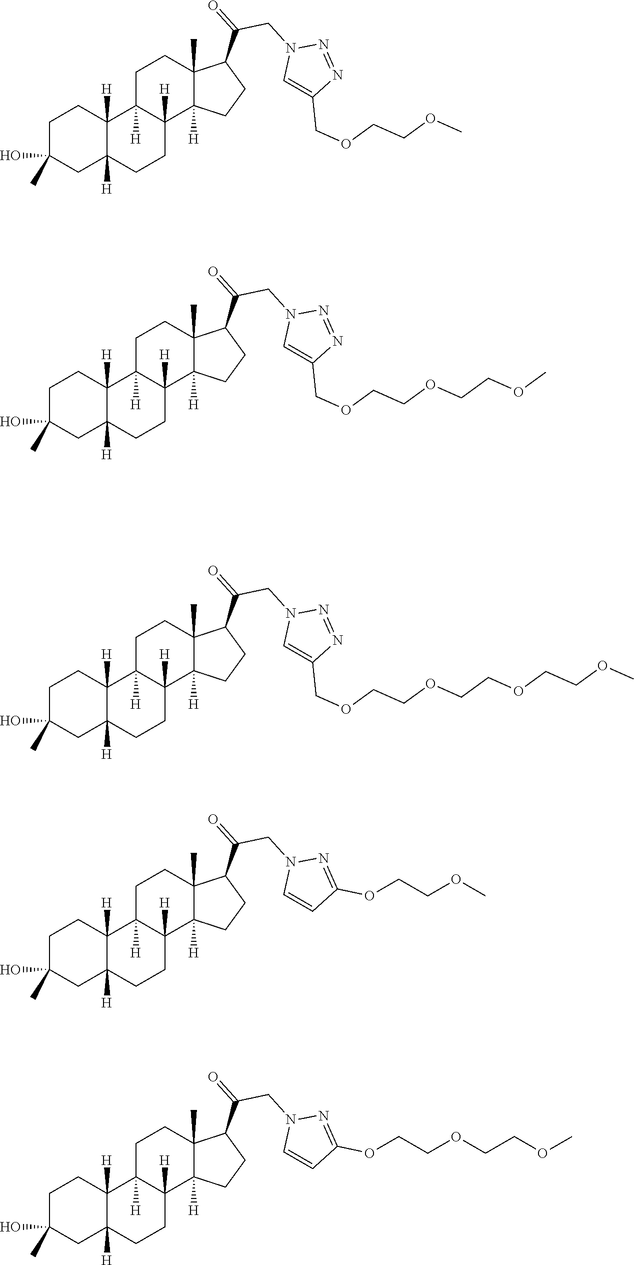

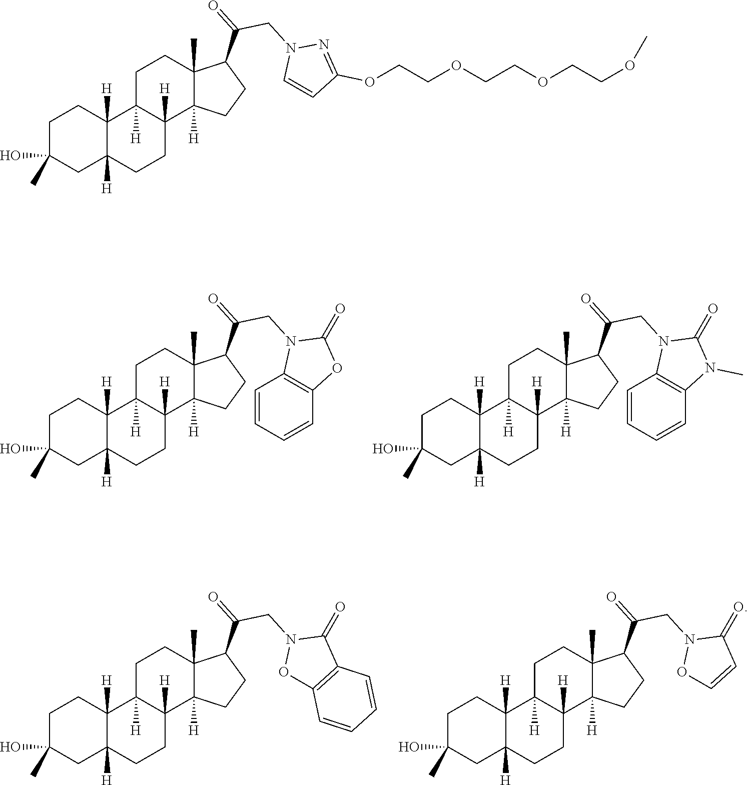

23. The compound of claim 1, having the structural formula:

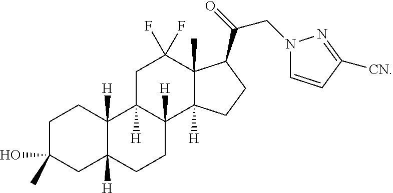

24. A pharmaceutical composition comprising a compound according to claim 1, effective to treat or reduce one or more diseases or disorders, in a mammal, including a human, and a pharmaceutically acceptable excipient, carrier, or diluent.

25-31. (canceled)

32. A unit dosage form comprising a pharmaceutical composition of claim 24.

33. (canceled)

34. A method for treating or reducing a disease or disorder, comprising administering to a subject in need thereof a pharmaceutical composition comprising a compound having the structural formula (I): 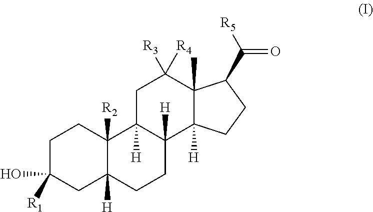
wherein
R₁ is H or a substituted or unsubstituted C₁-C₆ alkyl;
R₂ is H or a substituted or unsubstituted C₁-C₆ alkyl;
each of R₃ and R₄ is independently selected from the group consisting of H, halogen, a substituted or unsubstituted C₁-C₆ alkyl, optionally R₃ and R₄, along with the carbon to which they are attached, may form
an exocyclic double bond, or
a C₃-C₁₈-membered ring optionally substituted with one or more substituents selected from the group consisting of halogen, OH, CN, C₁-C₅ alkyl, and O—C₁-C₅ alkyl;
R₅ is OR′ or a C₁-C₆ alkyl optionally substituted with heterocyclic or heterobicyclic group, which is optionally substituted with one or more of CN, OH, halogen, a substituted or unsubstituted C₁-C₆ alkyl; provided that, if each of R₃ and R₄ is H, R₅ is a C₁ alkyl substituted with a heterocyclic or heterobicyclic group, which is optionally substituted with C═O(NR^{f}R^{g}), C(R^{f})(R^{g})(OR^{h}), or OR^{h}, wherein each of R′, R^{f} and R^{g} is independently selected from the group consisting of H, a substituted or unsubstituted C₁-C₆ alkyl, and R^{h} is (CH₂CH₂O)ₙCH₃ or CH₂O(CH₂CH₂O)ₙCH₃, wherein n is 1, 2, 3, 4 or 5,
or a pharmaceutically acceptable form or an isotope derivative thereof, effective to treat or reduce one or more of postpartum depression (PPD), major depressive disorder (MDD, or depression), insomnia, sleep apnea, restless legs syndrome, and narcolepsy, emotional disorders, depression, schizophrenia, bipolar disorder, obsessive-compulsive disorder, and other anxiety disorders, behavioral and pharmacological syndrome of dementia, and neurodegenerative diseases, or a related disease or disorder, in a mammal, including a human.

35-48. (canceled)
